SEQUENCE LISTING (3 sequences)

SEQ ID NO: 1 - DNA, 1525 bp, Streptomyces tendae
  source 1..1525: mol_type = genomic DNA
cacggagagtttgatcctggctcaggacgaacgctggcggcgtgcttaacacatgcaagtcgaacgatgaaccacttcggtggggattagtggcgaacgggtgagtaacacgtgggcaatctgccctgcactctgggacaagccctggaaacggggtctaataccggatactgaccctcacgggcatctgtgaggttcgaaagctccggcggtgcaggatgagcccgcggcctatcagcttgttggtgaggtaatggctcaccaaggcgacgacgggtagccggcctgagagggcgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggaagaagcgaaagtgacggtacctgcagaagaagcgccggctaactacgtgccagcagccgcggtaatacgtagggcgcgagcgttgtccggaattattgggcgtaaagagctcgtaggcggcttgtcacgtcggttgtgaaagcccggggcttaaccccgggtctgcagtcgatacgggcaggctagagttcggtaggggagatcggaattcctggtgtagcggtgaaatgcgcagatatcaggaggaacaccggtggcgaaggcggatctctgggccgatactgacgctgaggagcgaaagcgtggggagcgaacaggattagataccctggtagtccacgccgtaaacggtgggcactaggtgtgggcgacattccacgtcgtccgtgccgcagctaacgcattaagtgccccgcctggggagtacggccgcaaggctaaaactcaaaggaattgacgggggcccgcacaagcggcggagcatgtggcttaattcgacgcaacgcgaagaaccttaccaaggcttgacatacaccggaaagcattagagatagtgccccccttgtggtcggtgtacaggtggtgcatggctgtcgtcagctcgtgtcgtgagatgttgggttaagtcccgcaacgagcgcaacccttgtcccgtgttgccagcaagcccttcggggtgttggggactcacgggagaccgccggggtcaactcggaggaaggtggggacgacgtcaagtcatcatgccccttatgtcttgggctgcacacgtgctacaatggccggtacaatgagctgcgataccgcgaggtggagcgaatctcaaaaagccggtctcagttcggattggggtctgcaactcgaccccatgaagtcggagtcgctagtaatcgcagatcagcattgctgcggtgaatacgttcccgggccttgtacacaccgcccgtcacgtcacgaaagtcggtaacacccgaagccggtggcccaaccccttgtgggagggagctgtcgaaggtgggactggcgattgggacgaagtcgtaacaaggtagccgtaccggaaggtgcggctggatcacctccttt

SEQ ID NO: 2 - DNA, 20 bp, synthetic construct
  source 1..20: mol_type = other DNA; note = Forward primer 27F
agagtttgatcctggctcag

SEQ ID NO: 3 - DNA, 19 bp, synthetic construct
  source 1..19: mol_type = other DNA; note = Reverse primer 1492R
ggttaccttgttacgactt

(19) United States
(12) Patent Application Publication
(10) Pub. No.: US 2025/0176559 A1
(43) Pub. Date: Jun. 5, 2025

(54) Products And Methods For Improving Plant Growth Features

(71) Applicant: Aphea.Bio NV, Zwijnaarde (BE)

(72) Inventors: Tom Viaene, Gent (BE); Steven Vandenabeele, Oudenaarde (BE); Isabel Vercauteren, Woubrechtegem (BE); Sofie Goormachtig, Gent (BE)

(21) Appl. No.: 18/845,109

(22) PCT Filed: Mar. 10, 2023
(86) PCT No.: PCT/EP2023/056198

§ 371 Date: Sep. 9, 2024

(30) Foreign Application Priority Data
Mar. 10, 2022 (EP) 22161343.3

(51) Int. Cl. A01N 63/28; A01P 21/00; C12N 1/20; C12R 1/465

(52) U.S. Cl. CPC A01N 63/28; A01P 21/00; C12N 1/20; C12R 2001/465

(57) ABSTRACT

The application concerns methods for improving a plant growth feature, such as biomass, number of tillers per plant, and/or yield, of plants using certain bacteria or agricultural active compositions comprising the bacteria. Plants and plant parts treated with or heterologously disposed with said bacteria or compositions are also disclosed. Further, novel bacterial strains, and populations and agricultural active compositions comprising the same are provided.

### CROSS-REFERENCE TO RELATED APPLICATIONS

This application is a national phase entry under 35 U.S.C. § 371 of International Patent Application PCT/EP2023/056198, filed Mar. 10, 2023, designating the United States of America and published in English as International Patent Publication WO 2023/170281 on Sep. 14, 2023, which claims the benefit under Article 8 of the Patent Cooperation Treaty to European Patent Application Serial No. 22161343.3, filed Mar. 10, 2022, the entireties of which are hereby incorporated by reference.

### INCORPORATION BY REFERENCE

The ST.26 XML Sequence listing named “10590 US 2024 Sep. 9-Sequence Listing”, created on Mar. 10, 2023, and having a size of 8,192 bytes, is hereby incorporated herein by this reference in its entirety.

### FIELD OF THE INVENTION

The invention is broadly in the field of plant biology and bacterial strains, more precisely in the field of agricultural biologicals or agro-biologicals. In particular, the invention relates to products and methods for enhancing certain plant growth characteristics, and to a novel bacterial strain useful as a biostimulant.

### BACKGROUND OF THE INVENTION

There is a need for improved agricultural plants that will enable the food production demands with fewer resources and more environmentally sustainable inputs, for plants with improved responses to various biotic and abiotic stresses.

Crop performance is optimized primarily via technologies directed towards the interplay between crop genotype (e.g. plant breeding, genetically-modified (GM) crops) and its surrounding environment (e.g. fertilizer, synthetic herbicides, pesticides). While these paradigms have assisted in the increasing global food production, yield growth rates have stalled in many major crops. Shifts in the climate are linked to production instabilities as well as changing pest and disease pressures. In addition, genetically manipulated (GM) crops and agrochemicals have been challenged in their use in a large number of agricultural important crops and countries, resulting in a lack of acceptance for many GM traits and the exclusion of GM crops and many agrochemicals from global markets.

Therefore, there is an urgent need for novel solutions to crop improvement, more particularly, there is a need for innovative, effective, environmentally-sustainable, and publicly-acceptable approaches to improve the biomass, yield, and other agronomically important characteristics of plants.

A promising practice is the use of microorganisms that enhance plant growth and yield, increase tolerance to unfavorable conditions, and/or improve the resource use efficiency. In particular, a vast array of bacteria that live both within and around the plant tissues support the plant's health and growth.

Bacteria influence plant growth through multiple mechanisms, and in some cases through interactions with other bacteria. Specific bacterial strains inhabit various host plant tissues and have been isolated from plant leaves, stems, and roots. Several bacteria have been disclosed that increase plant growth and/or reduce susceptibility to diseases caused by fungi, bacteria, viruses or other plant pathogens.

The present invention aims to address at least some of the circumstances mentioned above. The aim of the invention is to provide further and/or improved means and methods to enhance agriculturally useful characteristics of an agricultural plant.

### SUMMARY OF THE INVENTION

The present invention is at least in part based on the inventors' discovery that certain bacteria can be used as an agricultural biological, in particular as a biostimulant, to increase one or more plant trait of agronomic importance, such as in particular the biomass, number of tillers per plant, and/or yield of a plant or part thereof.

As corroborated in the experimental section, which illustrates certain representative embodiments of the invention, the present inventors have found inter alia that plants grown from seeds treated with said bacteria (such as with said bacteria in a suspension, or with spores of said bacteria) demonstrated an increase in biomass per plant, such as dry biomass per plant, number of tillers per plant, and grain yield, as compared to untreated seeds.

Accordingly, an aspect of the invention relates to a method for improving a plant growth feature of a plant compared to an untreated plant, the method comprising administering bacteria of a bacterial strain which comprises a 16S polynucleotide having at least 99.34% sequence identity to SEQ ID NO: 1 (see Table 2) to the plant, a part thereof, a seed for growing the plant, or a locus of the plant. A related aspect provides the use of bacteria of a bacterial strain which comprises a 16S polynucleotide having at least 99.34% sequence identity to SEQ ID NO: 1 for improving a plant growth feature of a plant compared to an untreated plant. In certain preferred embodiments, biomass, number of tillers per plant, and/or yield of the plant may thereby be increased.

A further aspect relates to a method of treating a seed of a plant comprising inoculating the seed with said bacteria, such that the bacteria colonize a plant germinated from the inoculated seed and/or the soil or plant growth medium surrounding the growing plant, whereby the plant growth feature of the plant is improved compared to a plant germinated from an untreated seed. In certain preferred embodiments, biomass, number of tillers per plant, and/or yield of the plant may thereby be increased. Without wishing to be bound to any hypothesis, where the bacterial cells colonize the soil or plant growth medium in the vicinity of the plant roots, the bacteria may make accessible and provide to the plant absorbable nutrients.

Another aspect provides a plant or part thereof treated with said bacteria or with a composition comprising the bacteria; or a plant or part thereof heterologously disposed with said bacteria.

The inventors also identified a novel bacterial strain particularly advantageous in the present context. An aspect thus provides a bacterial strain as deposited under the Budapest Treaty at the Polish Collection of Microorganisms (PCM) under accession no. B/00365 on 11 Feb. 2022 with the following identification reference given to the deposited material by the depositor: TDC-B-A100D6, or a functional mutant thereof. For reasons of brevity, this strain (also see the particulars in Table 1) is also referred to as strain “TDC-B-A100D6” or strain “A100D6” throughout this specification.

Further aspects provide: a bacterial population comprising the aforementioned strain; as well as an agricultural active composition comprising the aforementioned strain.

The bacterial strains, products, methods, and uses of the present invention advantageously allow to improve one or more trait of agronomic importance in a plant, such as one or more plant growth features. Hence, the herein described bacterial strains provide several significant advantages to plants, in particular to agricultural plants, such as wheat, barley, maize, and the like. For example, dry biomass, wet biomass, number of tillers per plant, height of the plant, plant yield, seed yield, fruit yield, and/or emergence of a plant can be increased compared to untreated plants by applying the teachings of the present invention. Further the herein described bacterial strain also improves the ability of the plants to cope with abiotic stresses such as, but not limited to, drought. The present invention can thus allow to substitute or even abolish the use of chemical products such as fertilizers, and thereby advantageously facilitate more sustainable agriculture or increase the yield under adverse conditions. The teachings of the present invention can be immediately applied to any plant and, compared to provision of transgenic plants, do not require additional time for gene identification, generation and characterization of transgenic lines. Compared to the use of traditional agricultural methods including the application of chemical fertilizers, the present approaches can require less resources, can be less labor intensive, and are more environmentally friendly, and thereby also compatible with organic farming practices.

The above and further aspects and preferred embodiments of the invention are described in the following sections and in the appended claims. The subject-matter of appended claims is hereby specifically incorporated in this specification.

### DESCRIPTION OF THE DRAWINGS

The following description of the figures of specific embodiments of the invention is merely exemplary in nature and is not intended to limit the present teachings, their application or uses.

In FIG. 1, the graphs on the left visualize the values of the dry biomass or number of tillers per plant, with 95% confidence intervals for treated seeds and mock treated seeds in greenhouse condition, whereas the graphs on the right visualize the values of the difference between treated and mock treated seeds in dry biomass and number of tillers per plant with its 95% confidence interval in greenhouse condition. The percentage indicates the difference in dry biomass or number of tillers per plant expressed as a percentage of the mock treatment.

FIG. 1A represents graphs illustrating the increased dry biomass per plant at 6 weeks after sowing of wheat plants obtained from seeds treated with a formulation comprising spores of the A100D6 bacterial strain compared to the dry biomass per plant at 6 weeks after sowing of wheat plants obtained from untreated seeds (mock).

FIG. 1B represents graphs illustrating the increased dry biomass per plant at 6 weeks after sowing of wheat plants obtained from seeds treated with a formulation comprising living cells of the A100D6 bacterial strain compared to the dry biomass per plant at 6 weeks after sowing of wheat plants obtained from untreated seeds (mock).

FIG. 1C represents graphs illustrating the increased number of tillers per plant at 5 weeks after sowing of wheat plants obtained from seeds treated with a formulation comprising living cells of the A100D6 bacterial strain compared to the number of tillers per plant at 5 weeks after sowing of wheat plants obtained from untreated seeds (mock).

In FIG. 2 the graphs visualize agricultural field trials in, France, Belgium UK and Poland. Graphs on the left visualizes the values of winter wheat grain yield with 95% confidence intervals for treated seeds and untreated seeds, whereas the graph on the right visualizes the values of the difference between treated and untreated seeds in grain yield with its 95% confidence interval. The percentage indicates the difference in wheat grain yield expressed as a percentage of the untreated seeds.

FIG. 2A represents graphs illustrating the increased seed yield obtained from wheat seeds grown at one individual location, in France. The wheat plants were obtained from wheat seeds treated with a formulation comprising spores of the A100D6 bacterial strain and compared to wheat plants obtained from untreated wheat seeds (dashed line).

FIG. 2B represents graphs illustrating the increased seed yield obtained from wheat seeds grown at one individual location, in Belgium. The wheat plants were obtained from wheat seeds treated with a formulation comprising spores of the A100D6 bacterial strain and compared to wheat plants obtained from untreated wheat seeds (dashed line).

FIG. 2C represents graphs illustrating the increased seed yield obtained from wheat seeds grown at one individual location, in the UK. The wheat plants were obtained from wheat seeds treated with a formulation comprising spores of the A100D6 bacterial strain and compared to wheat plants obtained from untreated wheat seeds (dashed line).

FIG. 2D represents graphs illustrating the increased seed yield obtained from wheat seeds grown at one individual location, in Poland. The wheat plants were obtained from wheat seeds treated with a formulation comprising spores of the A100D6 bacterial strain and compared to wheat plants obtained from untreated wheat seeds (dashed line).

FIG. 3A visualizes the value in grain yield of winter wheat measured at a location in Poland in the season 2021-2022 with a 70% N fertilizer regime. The graph on the left visualizes the values of winter wheat grain yield with 95% confidence intervals for treated seeds and untreated seeds, whereas the graph on the right visualizes the values of the difference between treated and untreated seeds in grain yield with its 95% confidence interval. Wheat treated with a formulation comprising spores of the A100D6 bacterial strain and colorant showed an increased yield of 3.9% compared to the untreated seeds.

FIG. 3B visualizes the value in grain yield of winter wheat measured at a location in Bulgaria in the season 2021-2022 with a 70% N fertilizer regime. The graph on the left visualizes the values of winter wheat grain yield with 95% confidence intervals for treated seeds and untreated seeds, whereas the graph on the right visualizes the values of the difference between treated and untreated seeds in grain yield with its 95% confidence interval. Wheat treated with a formulation comprising spores of the A100D6 bacterial strain and colorant showed an increased yield of 4.1% compared to the untreated seeds.

FIG. 3C visualizes the value in grain yield of winter wheat measured at a location in Germany in the season 2021-2022 with a 100% N fertilizer regime. The graph on the left visualizes the values of winter wheat grain yield with 95% confidence intervals for treated seeds and untreated seeds, whereas the graph on the right visualizes the values of the difference between treated and untreated seeds in grain yield with its 95% confidence interval. Wheat treated with a formulation comprising spores of the A100D6 bacterial strain and colorant showed an increased yield of 3% compared to the untreated seeds.

FIG. 4. Average of the growth of the A100D6 bacterial strain in nutrient broth (NB) supplemented with different NaCl concentrations (1%; 5%; 10%; 20%; 25%) after a time period of 48 h.

### DETAILED DESCRIPTION OF THE INVENTION

As used herein, the singular forms “a”, “an”, and “the” include both singular and plural referents unless the context clearly dictates otherwise.

The terms “comprise”, “comprising”, “comprises” and “comprised of” as used herein are synonymous with “include”, “including”, “includes” or “contain”, “containing”, “contains”, and are inclusive or open-ended and do not exclude additional, non-recited members, elements or method steps. The terms also encompass “consisting of” and “consisting essentially of”.

The recitation of numerical ranges by endpoints includes all numbers and fractions subsumed within the respective ranges, as well as the recited endpoints. This applies to numerical ranges irrespective of whether they are introduced by the expression “from . . . to . . . ” or the expression “between . . . and . . . ” or another expression.

The terms “about” or “approximately” as used herein when referring to a measurable value such as a parameter, an amount, a temporal duration, and the like, are meant to encompass variations of and from the specified value, such as variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments of the invention described herein are capable of operation in other sequences than described or illustrated herein.

Whereas the term “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims. All documents cited in the present specification are hereby incorporated by reference in their entirety.

Throughout this disclosure, various publications, patents and published patent specifications may be referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation or meaning is meant to apply throughout this specification, i.e., also in the context of other aspects or embodiments of the invention, unless otherwise defined.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

By extensive experimental testing, the present inventors have found that certain bacterial strains exhibit plant growth promoting effects when administered to plants and hence that such strains can advantageously be used as biostimulants on plants. In particular, the bacterial strains provided an unexpected enhancement of plant biomass, number of tillers per plant, and/or yield, such as dry biomass, number of tillers per plant, and seed yield, in tested plants.

Accordingly, an aspect of the invention relates to a method for improving a plant growth feature of a plant compared to an untreated plant, the method comprising administering bacteria of a bacterial strain which comprises a 16S polynucleotide having at least 99.34% sequence identity to SEQ ID NO: 1 to the plant, a part thereof, a seed for growing the plant, or a locus of the plant. For example, such methods may be suitably practiced in the context of agriculture or horticulture.

Also provided is a method of treating a seed of a plant comprising inoculating the seed with said bacteria, such that the bacteria colonize a plant germinated from the inoculated seed and/or the soil or plant growth medium surrounding the growing plant, whereby the plant growth feature of the plant is improved compared to a plant germinated from an untreated seed.

A plant or part thereof treated with said bacteria or with a composition comprising the bacteria; or a plant or part thereof heterologously disposed with said bacteria, are also provided herein.

A further aspect discloses a bacterial strain as deposited under the Budapest Treaty at the Polish Collection of Microorganisms (PCM) under accession no. B/00365 on 11 Feb. 2022 with the following identification reference given to the deposited material by the depositor: TDC-B-A100D6, or a functional mutant thereof. The originally proposed taxonomic designation of this strain is Streptomyces tendae, as indicated on the deposit forms. However, the most recent taxonomy data indicates that the deposited strain may be more suitably classified as a Streptomyces marokkonensis strain. Also provided is a bacterial population comprising the aforementioned strain, as well as an agricultural active composition comprising the aforementioned strain.

As used herein, the term “bacterium”, “bacteria”, or “bacterial” refers in general to any prokaryotic organism, and may refer to an organism from either Kingdom Eubacteria (Bacteria), Kingdom Archaebacteria (Archaea), or both. In some cases, bacterial genera have been reassigned due to various reasons (such as, but not limited to, the evolving field of whole genome sequencing), and it is understood that such nomenclature reassignments are within the scope of any claimed genus.

As used herein, “bacterial strain” (which may be abridged to “strain” where the context makes clear that a bacterial strain is meant) refers to any of the prokaryotic microorganism belonging to the same class of species, including the species. The term “strain” as a basic operational unit of microbial taxonomy, such as bacterial taxonomy, is frequently used to denote a population made up of the descendants of a single isolation in pure culture, usually made up of a succession of cultures ultimately derived from an initial single colony. Where a species encompasses two or more distinct isolates, the term “strain” may be used to refer to an isolate or group of isolates that can be distinguished from other isolates of the same genus and species by phenotypic characteristics or genotypic characteristics or both.

In the practice of the present invention, the strain may be deemed as “isolated” or “purified”. The terms “isolated” or “purified” with reference to a particular component generally denote that such component exists in separation from—for example, has been separated from or prepared and/or maintained in separation from—one or more other components of its natural environment. The terms do not necessarily reflect the extent to which the component has been purified. Hence, the phrases “isolated bacterial strain” or “purified bacterial strain” may be seen as referring to a strain that has been removed from its natural milieu. In particular, the terms refer to substantially no other strains than the desired strain, which is thus substantially free of other contaminants, which can include microbial contaminants. Further, the terms may denote that the strain has been separated from materials with which it is normally found in nature. A strain heterologously disposed to other strains, or with compounds or materials with which it is not normally found in nature, is encompassed by the phrases “isolated bacterial strain” or “purified bacterial strain”.

In certain embodiments, the purified bacterial strains as taught herein may behave or be denoted as endophytes. An “endophyte” is an organism capable of living on a plant element (e.g., rhizoplane or phyllosphere) or within a plant element (e.g., endosphere) or on a surface in close physical proximity with a plant element (e.g., the rhizosphere or on a seed). Endophytes can occupy the intracellular or extracellular spaces of plant tissue, including but not limited to leaves, stems, flowers, fruits, seeds, or roots. An endophyte can be, for example, a bacterial or fungal organism, and can confer a beneficial property to the host plant such as an increase in yield, biomass, resistance, and/or fitness. An endophyte can be a bacterium or a fungus. As used herein, the term “microbe” or “strain” is sometimes used to describe an endophyte. As used herein, the microbes or strains as described herein can be labelled as endophytes.

Endophytes may favorably impact one or more traits of agronomic interest in plants. By means of an example and without limitation, a plant heterologously disposed with one or more endophyte microorganism, or a plant grown from a plant part or seed treated with or heterologously disposed with one or more endophyte microorganism, such as an endophytic bacterial or fungal strain, may exhibit a trait of agronomic interest, such as a trait selected from the group consisting of: disease resistance, drought tolerance, heat tolerance, cold tolerance, salinity tolerance, metal tolerance, herbicide tolerance, chemical tolerance, improved water use efficiency, improved phosphorus solubilization, improved phosphorus mobilization, improved nitrogen utilization, improved nitrogen fixation, pest resistance, herbivore resistance, pathogen resistance, increase in yield, increase in yield under water-limited conditions, health enhancement, vigor improvement, growth improvement, improved plant emergence, photosynthetic capability improvement, nutrition enhancement, altered protein content, altered oil content, increase in biomass, increase in number of tillers per plant, increase in shoot length, increase in root length, improved root architecture, increase in seed weight, altered seed carbohydrate composition, altered seed oil composition, increase in radical length, delayed senescence, stay-green, altered seed protein composition, increase in dry weight of mature plant reproductive elements, increase in fresh weight of mature plant reproductive elements, increase in number of mature plant reproductive elements per plant, increase in chlorophyll content, reduced number of wilted leaves per plant, reduced number of severely wilted leaves per plant, increase in number of non-wilted leaves per plant, improved plant visual appearance, and combinations thereof.

As used herein, a microorganism, such as a bacterial or fungal strain, such as an endophytic bacterial or fungal strain, is considered to have conferred an improved agricultural trait whether or not the improved trait arose from the plant, the strain, or the concerted action between the plant and the strain. Therefore, for example, where an improved agronomic trait results at least in part from the production of a beneficial hormone or chemical, for the purposes of the present specification the strain will be considered to have conferred the improved agronomic trait upon the plant as compared to a plant, plant part or seed that has not been treated with or heterologously disposed with said strain, whether the beneficial hormone or chemical is produced by the plant or by the strain.

Particularly envisaged herein is the administration of live bacteria and/or microorganisms. The term “live” as used herein is synonymous with “viable” and refers to any living intact state of a microorganism, such as active growth or dormancy, from which state it can multiply and/or reproduce itself in a medium capable of supporting the growth of the microorganism. For sporulating bacteria and/or microorganisms, the spores and their use are also encompassed by the present teaching. Typically, substantially all bacteria or microorganisms comprised by populations or compositions intended herein may be live or viable. For example, at least 50%, preferably at least 60%, more preferably at least 75%, still more preferably at least 90%, such as at least 95%, 96%, 97%, 98%, 99% or 100% of the bacteria or microorganisms in the population or composition may be viable, such as capable of forming colonies when plated on a suitable solid medium.

The term “16S polynucleotide”, or synonymous terms such as “16S nucleotide sequence” or “16S”, refer to the nucleic acid sequence, such as in particular the DNA sequence, of the 16S ribosomal RNA (rRNA) of a bacterium. 16S rRNA gene sequencing is a well-established method for studying phylogeny and taxonomy of bacteria. A full length 16S nucleic acid sequence is approximately 1500 nucleotides in length. In certain embodiments, the bacterial strain may comprise a single copy of the 16S rRNA gene. In certain embodiments, the bacterial strain may comprise more than one copy of the 16S rRNA gene, such as two, three or more (multicopy) copies of the 16S rRNA gene. In such embodiments where two or more 16S rRNA gene copies are found in the bacterial strain, at least one of these 16S rRNA gene copies complies with the sequence identity requirements specified in the present application, preferably two or more and preferably all of the 16S rRNA gene copies (each independently) comply with the stated sequence identity requirements. By means of an example and without limitation, where a bacterial strain comprises three 16S rRNA gene copies, at least one, preferably at least two, and more preferably all three 16S rRNA gene copies will, each independently, display at least 99.75% sequence identity to SEQ ID NO: 1, and may in certain preferred embodiments be identical. Conveniently, the 16S rRNA sequence can be determined by sequencing (e.g., Sanger sequencing) the 16S gene sequence(s) in the chromosomal DNA, which may be amplified (e.g., PCR amplified) using suitable amplification primers, such as in particular the Forward primer 27F: AGAGTTTGATCCTGGCTCAG (SEQ ID NO: 2) and Reverse primer1492R: GGTTACCTTGTTACGACTT (SEQ ID NO: 3).

The terms “identity”, “sequence identity” or “identical” in the context of nucleotide sequences may be used interchangeably herein, and refer to the extent that nucleic acid sequences are identical on a nucleotide-by-nucleotide basis, over a window of comparison. The percentage of sequence identity may be calculated by comparing two optimally aligned sequences over the window of comparison, determining the number of positions at which the identical nucleic acid base occurs in both sequences to yield the number of matched positions, dividing the number of matched positions by the total number of positions in the window of comparison, and multiplying the result by 100 to yield the percentage of sequence identity. The percent identity value may, but need not, be rounded to the nearest tenth. For example, 98.11, 98.12, 98.13, and 98.14 may be rounded down to 98.1, while 98.15, 98.16, 98.17, 98.18, and 98.19 may be rounded up to 98.2.

Sequence identity between nucleic acids as envisaged herein may be determined using suitable algorithms for performing sequence alignments and determination of sequence identity as know per se. Exemplary but non-limiting algorithms include those based on the Basic Local Alignment Search Tool (BLAST) originally described by Altschul et al. 1990 (J Mol Biol 215:403-10), such as the “Blast 2 sequences” tool described by Tatusova and Madden 1999 (FEMS Microbiol Lett 174:247-250), or the “blastn suite-2sequences” sequence alignment algorithm described by Zheng Zhang et al. 2000 (J Comput Biol 2000, vol. 7 (1-2), 203-14), now incorporated into the BLAST program suite available at ncbi.nlm.nih.gov. The skilled person can implement such algorithms and set the requisite parameters. By means of an example and without limitation, parameters for the BLASTN program may be as follows: cost to open a gap=0, cost to extend a gap=2.5, reward for a match=1, penalty for a mismatch=−2, Expect value=0.05, word size=28, Low Complexity Filter=Yes. There are further algorithms known in the art that can be used to measure nucleotide sequence identity. Nucleotide sequence identity can be measured by a local or global alignment, preferably implementing an optimal local or optimal global alignment algorithm. For example, a global alignment may be generated using an implementation of the Needleman-Wunsch algorithm (Needleman & Wunsch. Journal of Molecular Biology 1970, vol. 48 (3), 443-53). For example, a local alignment (which does not consider the entirety of the sequence but tries to find the longest subsequence that confirms to a given matching criteria) may be generated using an implementation of the Smith-Waterman algorithm (Smith & Waterman Journal of Molecular Biology 1981, vol. 147 (1), 195-197). Optimal global alignments using the Needleman-Wunsch algorithm and optimal local alignments using the Smith-Waterman algorithm are implemented in USEARCH (https://www.drive5.com/usearch/), for example USEARCH version 11.0.667.

A gap is a region of an alignment wherein a sequence does not align to a position in the other sequence of the alignment. In global alignments, terminal gaps are discarded before identity is calculated. For both local and global alignments, internal gaps are counted as differences. A terminal gap is a region beginning at the end of a sequence in an alignment wherein the nucleotide in the terminal position of that sequence does not correspond to a nucleotide position in the other sequence of the alignment and extending for all contiguous positions in that sequence wherein the nucleotides of that sequence do not correspond to a nucleotide position in the other sequence of the alignment.

Sequence identity as envisaged herein in particular denotes overall sequence identity, i.e., sequence identity calculated from optimally aligning the whole sequences of the to-be-compared 16S rRNA genes. In other words, the nucleic acid sequences to be aligned are the complete 16S rRNA genes, and the window of comparison corresponds to the whole region of optimal alignment between these complete 16S sequences, i.e., to the alignment length. Hence, in an example, a query 16S rRNA gene sequence, such as the sequence set forth in SEQ ID NO: 1, is optimally aligned with another 16S rRNA gene sequence (in case of a global alignment any terminal gaps are disregarded; in case of a local alignment the region of alignment will be expressed as the region between a given 5′ and a given 3′ position in the query sequence), and the percentage sequence identity is calculated over a window of comparison which corresponds to the whole region of alignment, counting internal gaps as differences.

Bacterial strains as envisaged herein preferably comprise a 16S polynucleotide the length of which is between 90% and 110% (1373-1678 nucleotides), more preferably between 95% and 105% (1449-1601 nucleotides) of the length of the 16S region polynucleotide shown in SEQ ID NO: 1. These 16S sequences can be subjected to pairwise sequence comparisons with SEQ ID NO: 1.

When two complete 16S region sequences in a pairwise sequence comparison are optimally aligned, for example by a local alignment algorithm such as BLAST, it is particularly envisaged that the alignment length is at least 90% of the length of the shorter one of the two 16S sequences, preferably at least about 91%, 92%, 93%, 94%, more preferably at least about 95%, or at least about 96%, 97%, 98%, 99% or 100% of the length of the shorter one of the two 16S sequences, and the window of comparison corresponds to the whole alignment length.

Preferably, the region of alignment, for example the region of alignment provided by a local alignment algorithm such as BLAST, will comprise at least 90% of the length of SEQ ID NO: 1, more preferably at least about 91%, 92%, 93%, 94%, even more preferably at least about 95%, or at least about 96%, 97%, 98%, 99% or 100% of the length of SEQ ID NO: 1. Preferably, the region of alignment, for example the region of alignment provided by a local alignment algorithm such as BLAST, will comprise at least 1373 contiguous (the term contiguous in this context does not exclude the presence of internal gaps in the alignment) nucleotides of SEQ ID NO: 1, more preferably at least 1400 contiguous nucleotides, such as at least 1410, at least 1420, at least 1430, or at least 1440 contiguous nucleotides of SEQ ID NO: 1. Particularly preferably, the region of alignment will comprises at least 1449, or in increasing order of preference, at least 1450, at least 1460, at least 1470, at least 1480, at least 1490, or at least 1500, or at least 1510, or at least 1520, or all 1525 contiguous nucleotides of SEQ ID NO: 1.

In certain preferred embodiments, the bacterial strain comprises a 16S polynucleotide having at least 99.41% sequence identity to SEQ ID NO: 1, or in increasing order of preference, at least 99.48%, at least 99.54%, at least 99.61%, at least 99.67%, at least 99.74%, at least 99.80%, at least 99.87%, or at least 99.93% sequence identity to SEQ ID NO: 1. In certain still more preferred embodiments, the bacterial strain comprises a 16S polynucleotide having 100.00% sequence identity to SEQ ID NO: 1.

In certain particularly preferred embodiments, the bacterial strain comprises a 16S polynucleotide which, when aligned over a region of alignment comprising at least 1449 contiguous nucleotides of SEQ ID NO: 1, or in increasing order of preference, over a region of alignment comprising at least 1450, at least 1460, at least 1470, at least 1480, at least 1490, at least 1500, at least 1510, at least 1520, or all 1525 contiguous nucleotides of SEQ ID NO: 1, will display no more than 10, preferably in increasing order of preference no more than 9, 8, 7, 6, 5, 4, 3, or 2, yet more preferably no more than 1, and most preferably no nucleotide mismatches and internal gaps with SEQ ID NO: 1. The mismatch or internal gap in this context refers to a single nucleotide mismatch or a gap that involves or spans a single nucleotide.

In certain particularly preferred embodiments, the bacterial strain comprises a 16S polynucleotide as set forth in (i.e., identical to) SEQ ID NO: 1, i.e., a 16S polynucleotide identical over the full length thereof to SEQ ID NO: 1.

In certain embodiments, the bacterial strain is a Streptomyces tendae or a Streptomyces marokkonensis strain.

In certain embodiments, the bacterial strain as envisaged herein is the strain as deposited under the Budapest Treaty at the Polish Collection of Microorganisms (PCM) under accession no. B/00365 on 11 Feb. 2022 with the following identification reference given to the deposited material by the depositor: TDC-B-A100D6, or a functional mutant thereof.

The term “functional mutant” means a bacterial strain directly or indirectly obtained by genetic modification (such as by random mutagenesis or by targeted genetic modification) of the respective referenced strain and retaining at least some extent of the activity of the referenced strain on the plant growth feature of interest, such as ability to or activity in increasing the biomass, number of tillers per plant, and/or yield of the plant, preferably retaining at least 10%, such as at least 20%, at least 30%, or at least 40%, preferably at least 50%, such as at least 60%, at least 70%, or at least 80%, more preferably at least 90%, such as 100%, or even greater than 100% of the activity of the referenced strain on the plant growth feature of interest. The genetic modification of a functional mutant can be achieved through any means, such as, but not limited to, chemical mutagens, ionizing radiation, transposon-based mutagenesis, or via conjugation, transduction, or transformation using the referenced strains as either the recipient or donor of genetic material. In certain embodiments, the 16S rRNA gene sequence of the functional mutant remains identical to the 16S rRNA gene sequence of the reference strain. Hence, the functional mutant may preferably comprise the 16S rRNA gene sequence as shown in SEQ ID NO: 1. In certain embodiments, the functional mutant comprises at most 10, such as in increasing order of preference, at most 9, 8, 7, 6, 5, 4, 3, 2, or at most 1 chromosomal loci (such as genes) whose nucleic acid sequence differs from (has been modified compared to) the sequence of the corresponding loci in the reference strain, and/or the functional mutant comprises at most 10, such as in increasing order of preference, at most 9, 8, 7, 6, 5, 4, 3, 2, or at most 1 gene of the functional mutant carries a non-synonymous mutation not present in the reference strain. Hence, also disclosed is a method of genetically modifying the bacterial strain as envisaged herein, such as by mutagenesis, for example random or directed mutagenesis, and/or by transgenesis, for example by the introduction of one or more transgenes thereto, whereby a functional mutant of the referenced strain is obtained.

In certain embodiments, bacteria of two or more bacterial strains as described in the present specification may be administered to the plant, the part thereof, the seed for growing the plant, or the locus of the plant.

In certain embodiments, the bacteria as described in the present specification may be administered to the plant, the part thereof, the seed for growing the plant, or the locus of the plant in conjunction with one or more additional plant-beneficial microorganism. As used throughout the present specification, the term “microorganism” or “microbe” refers to any strain, any species or taxon of microorganism, including, but not limited to, archaea, bacteria, microalgae, fungi (including mold and yeast species), mycoplasmas, microspores, nanobacteria, oomycetes, and protozoa. In some embodiments, a microbe or microorganism is a bacterial strain. In some embodiments, a microbe or microorganism is a fungal strain. In some embodiments, a microbe or microorganism is an endophyte, for example a bacterial or fungal endophyte, which is capable of living within a plant. In some embodiments, a microbe or microorganism encompasses individual cells (e.g., unicellular microorganisms) or more than one cell (e.g., multi-cellular microorganism).

Diverse plant-associated microorganisms can positively impact plant health and physiology in a variety of ways. In particular, plant-beneficial microorganisms, when administered to a plant, plant part, a seed for growing a plant, or a locus of a plant may improve one or more traits of agronomic importance in a plant, such as one or more plant growth features. Where the improved trait can be quantified, any extent of an improvement is contemplated. For example, a plant-beneficial microorganism may provide an improved trait of agronomic importance in a plant that is of at least 3%, between 3% and 5%, at least 5%, between 5% and 10%, least 10%, between 10% and 15%, for example at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, at least 100%, between 100% and 150%, at least 150%, between 150% and 200%, at least 200%, between 200% and 300%, at least 300% or more, when compared with a reference plant grown under the same conditions. By means of an illustration, a plant-beneficial microorganism may be capable of increasing nutrient uptake and/or nutrient use efficiency of a treated plant as compared to an untreated plant, increasing the nitrogen fixating capacities or phosphorus uptake of a treated plant as compared to an untreated plant, increasing the amount of biomass of a treated plant as compared to an untreated plant, increasing the number of tillers per plant of a treated plant as compared to an untreated plant, increasing growth and/or yield of a treated plant as compared to an untreated plant, and/or helping a treated plant overcome stress conditions, such as nutrient stress, compared to an untreated plant; and the like. Further traits of agronomic importance that can be improved by plant-beneficial microorganisms may include disease resistance, drought tolerance, heat tolerance, cold tolerance, salinity tolerance, metal tolerance, herbicide tolerance, chemical tolerance, improved water use efficiency, improved phosphorus solubilization, improved phosphorus mobilization, improved nitrogen utilization, improved nitrogen fixation, pest resistance, herbivore resistance, pathogen resistance, increase in yield, increase in yield under water-limited conditions, health enhancement, vigor improvement, growth improvement, improved plant emergence, photosynthetic capability improvement, nutrition enhancement, altered protein content, altered oil content, increase in biomass, increase in number of tillers per plant, increase in shoot length, increase in root length, improved root architecture, increase in seed weight, altered seed carbohydrate composition, altered seed oil composition, increase in radical length, delayed senescence, stay-green, altered seed protein composition, increase in dry weight of mature plant reproductive elements, increase in fresh weight of mature plant reproductive elements, increase in number of mature plant reproductive elements per plant, increase in chlorophyll content, reduced number of wilted leaves per plant, reduced number of severely wilted leaves per plant, increase in number of non-wilted leaves per plant, and/or improved plant visual appearance, and the like.

By means of an illustration and without limitation, plant-beneficial microorganisms may include mycorrhizal fungi, including endomycorrhizal fungi and ectomycorrhizal fungi, such as fungi belonging to the divisions Basidiomycota, Ascomycota, and Zygomycota, bacteria of the family Rhizobiaceae, bacteria of the genera Frankia, Azotobacter, Azospirillum, Acetobacter, Azoarcus, Burkholderia, Herbaspirillum, Pseudomonas (e.g., Pseudomonas fluorescens, P. putida, P. gladioli), Bacillus (Bacillus subtilis, B. cereus, B. circulans), further bacteria such as Serratia marcescens, Flavobacterium spp., Alcaligenes sp., Agrobacterium radiobacter, and others. In certain embodiments, the plant-beneficial microorganisms are selected from those disclosed in WO2018060519, WO2020161351, and WO2020161352.

In certain embodiments, the one or more additional plant-beneficial microorganism may be selected to improve the efficacy of the bacterial strain as taught herein, in particular efficacy in improving the plant growth feature acted on by the bacterial strain. Hence, also provided is a method of improving the efficacy of the bacterial strain as taught herein, comprising the selection of an additional plant-beneficial microorganism, whereby co-administration of the additional plant-beneficial microorganism with the bacterial strain or strains to a plant, plant part, or seed improves the plant growth feature. In such embodiments, the administration of the plant-beneficial microorganism alone may but need not lead to an improvement in a plant trait.

In certain embodiments, the bacteria can be comprised in or be part of an agricultural active composition. Hence, the methods may entail administering or applying such an agricultural active composition to the plant, the part thereof (e.g., roots), the seed for growing the plant, or the locus of the plant (e.g., to soil or plant growth medium surrounding the plant).

The term “composition” generally refers to a thing composed of two or more components, and more specifically denotes a combination or mixture of two or more materials, such as elements, molecules, substances, and/or microorganisms, as well as reaction products and decomposition products formed from the materials of the composition. The term may be interchangeably used with the terms “formulation” or “preparation”.

Agricultural active compositions typically comprise one or more agriculturally active ingredients and one or more agriculturally acceptable carrier or auxiliary. The terms “active ingredient” or “active component” can be used interchangeably and broadly refer to a material, such as an element, molecule, substance, and/or microorganism, which, when provided in an effective amount, achieves a desired outcome, such as achieves one or more effects on one or more traits of agronomic importance in plants. Typically, an active ingredient as intended herein may achieve such outcome(s) through interacting with and/or modulating the plant, part thereof, a seed for growing the plant, or the locus of the plant. The terms “agriculturally acceptable” or “agriculturally compatible” are consistent with the art and mean not deleterious to the recipient plant, such as not producing, having or causing any adverse effects when applied to a plant or to an organ, part or element of the plant, or adverse effects to the plant grown from that plant organ, part or element. The agriculturally active formulations may comprise materials which facilitate or enhance the stability, viability, storage, and/or administration of the bacterial strain(s) and/or plant-beneficial microorganism(s) as disclosed herein, and/or the colonization of the plant thereby.

Compositions as typically used herein may be liquid, semi-solid, or solid, and may include solutions or dispersions. Non-limiting examples of the compositions as taught herein may be soluble powders, soluble granules, wettable granules, tablet formulations, dry flowables, aqueous flowables, wettable dispersible granules, oil dispersions, suspension concentrates, dispersible concentrates, emulsifiable concentrates, aqueous suspensions, fertilizer granules, sprayables, and the like. In certain embodiments, a composition may be composed of components that are provided to an end user as a mixture, i.e., the composition components are already admixed. In certain embodiments, a composition may be composed of components one or more of which are provided to an end user in a physically separated form (e.g., in separate containers or vials) from one or more other components of the composition, although typically as part of the same product package or dispensing device. By means of an example and without limitation, the composition may comprise one or more components provided in one container, and one or more components provided in another container. Such arrangement allows the end user to admix the components of the composition shortly before use. For example, the composition may comprise the bacteria and optionally further plant beneficial microorganism(s) as taught herein provided in one container, and one or more auxiliaries provided in another container, to be admixed by the end user before use.

In certain embodiments, the composition comprises one or more agriculturally acceptable auxiliary. The terms “auxiliary”, “auxiliary agent”, “additive”, or “adjuvant” may be used interchangeably herein. The auxiliaries may be natural or synthetic organic or inorganic materials which facilitate the administration of actives to plants, plant parts, seeds, or plant growth loci. In certain embodiments, the auxiliaries may be one or more of as a solvent, a carrier, a surfactant, a sticker, an antifreeze agent, a thickener, a buffering agent, an antifoaming agent, an antioxidant, a preservative, an aroma, or a colorant. Suitable auxiliary agents and inert agents are known in the art and are commercially available. In general, the bacteria and optionally further plant-beneficial microorganism(s) can be combined with any solid, semi-solid or liquid additive customarily used for formulation purposes. A carrier is to be understood as meaning a natural or synthetic, organic or inorganic substance which is mixed or combined with the bacteria and optionally further plant-beneficial microorganism(s) for better applicability, in particular for application to plants or plant parts such as seeds. The carrier, which may be solid, semi-solid, or liquid, is generally inert and suitable for use in agriculture or horticulture. For instance, liquid carriers may include water, organic solvents, and mineral oils and vegetable oils. Suitable liquefied gaseous extenders or carriers are liquids which are gaseous at ambient temperature and under atmospheric pressure, for example aerosol propellants, such as butane, propane, nitrogen and carbon dioxide. A sticker is to be understood as meaning an additive or adjuvant to improve adhesive properties of the composition to the plant or part thereof. Suitable surfactants are emulsifiers, dispersants or wetting agents having ionic or nonionic properties, or mixtures of these surfactants. It is possible to use colorants such as inorganic pigments, for example iron oxide, titanium oxide, Prussian blue, and organic dyes, such as alizarin dyes, azo dyes and metal phthalocyanine dyes, and trace nutrients, such as salts of iron, manganese, boron, copper, cobalt, molybdenum and zinc. Stabilizers, such as low-temperature stabilizers, preservatives, antioxidants, light stabilizers or other agents which improve chemical and/or physical stability may also be present.

In certain embodiments, the bacteria and optionally further plant-beneficial microorganism(s) may be administered in combination with one or more other non-active or active ingredients that are non-toxic thereto. Such other ingredients may be an oil, an emulsifier, a spreader, a cryoprotectant, a binder, a dispersant, a surfactant, a buffer, a tackifier, a stabilizer, a microbial stabilizer, a bactericide (e.g., effective against bacteria other than those administered), a fungicide, a complexing agent, a herbicide, a nematicide, an insecticide, a molluscicide, an algicide, a fertilizer, a micronutrient fertilizer material, a plant growth regulator, a rodenticide, a preservative, a polymer, a desiccant, a nutrient, an excipient, a wetting agent, a salt, or any combination thereof.

In certain embodiments, the bacteria and optionally further plant-beneficial microorganism(s) may be co-administered and/or co-formulated with further biologicals or agrochemicals that stimulate plant growth and/or yield. In certain embodiments, particular strains may be selected on the basis of their compatibility with commonly used biologicals or agrochemicals. Plants, particularly agricultural plants, can be treated with a vast array of biologicals or agrochemicals. In some cases, particular strain may be selected to be compatible with biologicals or agrochemicals with complexing properties, to facilitate persistence of the strain in the plant. There also exist many complexing agents that do not penetrate the plant, at least at a concentration sufficient to interfere with the administered bacteria. Where a systemic complexing agent is used in the plant, compatibility of the strain to be inoculated with such agents may be an important variable to consider. In an embodiment, purified bacterial strains that are compatible with biologicals or agrochemicals can be used to inoculate plants, plant elements or growth media according to the methods described herein.

Bactericide-compatible strains can also be isolated by selection on liquid medium. The culture of strains can be plated on petri dishes without any forms of mutagenesis; alternatively, strains can be mutagenized using any means known in the art. For example, strain cultures can be exposed to UV light, gamma-irradiation, or chemical mutagens such as ethylmethanesulfonate (EMS), ethidium bromide (EtBr), dichlorvos (DDVP), methyl methane sulphonale (MMS), triethylphosphate (TEP), trimethylphosphate (TMP), nitrous acid, or DNA base analogs, prior to selection on bactericide comprising media. Alternatively or in addition, where the mechanism of action of a particular bactericide is known, the target gene can be specifically mutated (either by gene deletion, gene replacement, site-directed mutagenesis, etc.) to generate a strain that is resilient against that particular chemical. The above-described methods can be used to isolate strains that are compatible with both bacteriostatic and bactericidal compounds. The biological or agrochemical compatible strains generated can be detected in samples. For example, where a transgene was introduced to render the strain compatible with the biological(s) or agrochemical(s), the transgene can be used as a target gene for amplification and detection by PCR. In addition, where point mutations or deletions to a portion of a specific gene or a number of genes results in compatibility with the biological(s) or agrochemical(s), the unique point mutations can likewise be detected by PCR or other means known in the art. Such methods allow the detection of the strain even if it is no longer viable.

In certain embodiments, the composition is a liquid composition. The compositions may be a ready-to-use composition which can be administered or applied with a suitable apparatus, or the composition may be a concentrate or a concentrated formulation which is to be diluted in a solvent, such as water or an aqueous solution or buffer prior to use. In certain further embodiments, the composition is an aqueous composition. In certain embodiments, the composition is a sprayable liquid or a concentrate. In certain embodiments the composition is a spray, a sprayable liquid or a dip.

The compositions as intended herein can encompass an effective amount of the bacteria and optionally further plant-beneficial microorganism(s), i.e., an amount sufficient to elicit the desired outcome, such as one or more effects on one or more traits of agronomic importance in plants, such as an increase in the biomass of a plant, or yield of a plant, or both biomass and yield of a plant compared to an untreated plant, that is being sought by the user, in either a single or multiple doses, preferably in a single dose.

In certain embodiments, the composition comprises the bacteria at a concentration of at least about 10 CFU/ml. In certain embodiments, the composition comprises the bacteria at a concentration of at least about 10² CFU/ml. As used herein, a “colony forming unit” or “CFU” refers to a measure of viable microorganisms in a sample. A CFU is an individual viable cell capable of forming on a solid medium a visible colony whose individual cells are derived by cell division from one parental cell. The phrases “CFU”, “CFU/ml”, and “CFU/g”, herein and throughout the specification, also encompass the reference to “spores”, “spores/ml” or “spores/g”, respectively, in case the microorganism lends itself to being administered in the form of spores.

In certain embodiments, the liquid composition comprises the bacteria at a concentration of at least about 10² CFU/ml. In certain embodiments, the liquid composition comprises the bacteria at a concentration of at least about 10³ CFU/ml, at least about 10⁴ CFU/ml, at least about 10⁵ CFU/ml, at least about 10⁶ CFU/ml, at least about 10⁷ CFU/ml, at least about 10⁸ CFU/ml, at least about 10⁹ CFU/ml, at least about 10¹⁰ CFU/ml, at least about 10¹¹ CFU/ml, or at least about 10¹² CFU/ml.

In certain embodiments, the liquid composition comprises the bacteria at a concentration of from 1×10² CFU/ml to 1×10¹² CFU/ml, or from 1×10³ CFU/ml to 1×10¹¹ CFU/ml, or from 1×10³ CFU/ml to 1×10¹⁰ CFU/ml, or from 1×10⁴ CFU/ml to 1×10¹⁰ CFU/ml, or from 1×10⁵ CFU/ml to 1×10¹⁰ CFU/ml, or from 1×10⁶ CFU/ml to 1×10¹⁰ CFU/ml, or from 1×10⁶ CFU/ml to 1×10⁹ CFU/ml, or from 1×10⁷ CFU/ml to 1×10¹⁰ CFU/ml, or from 1×10⁷ CFU/ml to 1×10⁹ CFU/ml, or from 1×10⁸ CFU/ml to 1×10¹⁰ CFU/ml, or from 1×10⁸ CFU/ml to 1×10⁹ CFU/ml.

In certain embodiments, the composition is a non-liquid composition. In certain preferred embodiments, the composition may be a solid composition or a powdered composition. In certain preferred embodiments, the composition is a powder. The term “powder” refers to a dry, bulk solid composed of many very fine particles that may flow freely when shaken or tilted.

In certain embodiments, the non-liquid composition, such as the powder, comprises the bacteria at an amount of at least about 10 CFU/g. In certain embodiments, the non-liquid composition, such as the powder, comprises the bacteria at an amount of at least about 10² CFU/g. In certain embodiments, the non-liquid composition comprises the bacteria at an amount of at least about 10² CFU/g. In certain embodiments, the non-liquid composition comprises the bacteria at an amount of at least about 10³ CFU/g, at least about 10⁴ CFU/g, at least about 10⁵ CFU/g, at least about 10⁶ CFU/g, at least about 10⁷ CFU/g, at least about 10⁸ CFU/g, at least about 10⁹ CFU/g, at least about 10¹⁰ CFU/g, at least about 10¹¹ CFU/g, or at least about 10¹² CFU/g.

In certain embodiments, the non-liquid composition comprises the bacteria at an amount of from 1×10² CFU/g to 1×10¹² CFU/g, or from 1×10³ CFU/g to 1×10¹¹ CFU/g, or from 1×10³ CFU/g to 1×10¹⁰ CFU/g, or from 1×10⁴ CFU/g to 1×10¹⁰ CFU/g, or from 1×10⁵ CFU/g to 1×10¹⁰ CFU/g, or from 1×10⁶ CFU/g to 1×10¹⁰ CFU/g, or from 1×10⁶ CFU/g to 1×10⁹ CFU/g, or from 1×10⁷ CFU/g to 1×10¹⁰ CFU/g, or from 1×10⁷ CFU/g to 1×10⁹ CFU/g, or from 1×10⁸ CFU/g to 1×10¹⁰ CFU/g, or from 1×10⁸ CFU/g to 1×10⁹ CFU/g.

In certain embodiments, the composition may comprise bacteria of two or more bacterial strains as described in the present specification. In certain embodiments, the composition may comprise at least about 10² CFU/ml or at least about 10² CFU/g-such as the aforementioned more specific CFU/ml or CFU/g amount ranges—of bacteria of all the strains collectively or preferably of each of the strains individually and independently.

In certain embodiments, the composition comprises the one or more optional further plant-beneficial microorganism, such as cells or spores of one or more plant-beneficial microorganism strain, at a concentration or an amount, collectively or each individually and independently, of at least about 10² CFU/ml or 10² CFU/g, at least about 10³ CFU/ml or CFU/g, at least about 10⁴ CFU/ml or CFU/g, at least about 10⁵ CFU/ml or CFU/g, at least about 10⁶ CFU/ml or CFU/g, at least about 10⁷ CFU/ml or CFU/g, at least about 10⁸ CFU/ml or CFU/g, at least about 10⁹ CFU/ml or CFU/g, or at least about 10¹⁰ CFU/ml or CFU/g. More preferably, the composition comprises the one or more optional further plant-beneficial microorganism, such as cells or spores of one or more plant-beneficial microorganism strain, at a concentration or an amount, collectively or each individually and independently, of between 10³ to 10¹⁰ CFU/ml or CFU/g, between 10⁴ to 10¹⁰ CFU/ml or CFU/g, between 10⁵ to 10¹⁰ CFU/ml or CFU/g, between 10⁶ to 10¹⁰ CFU/ml or CFU/g, between 10⁶ to 10⁹ CFU/ml or CFU/g, between 10⁷ to 10⁹ CFU/ml or CFU/g, or between 10⁸ to 10⁹ CFU/ml or CFU/g.

In certain embodiments, the bacteria may be comprised by and administered as part of a bacterial population. A bacterial population may comprise bacteria of one or more bacterial strains as described in the present specification, such as of two, three or more strains as described in the present specification.

More generally, a bacterial population may comprise one or more, preferably two or more (e.g., 3, 4, 5, 6, 7, 8, 9, 10, 15, 20, 25 or more than 25) purified bacterial strains, wherein the strains may originate from different families of bacteria, or different genera of bacteria, or from the same genera but different species of bacteria. The taxonomically different bacterial strains can be obtained from the same cultivar of plant, different cultivars of the same plant, or different species of the same type of plant. The bacterial strains can be obtained from the soil wherein the plant is grown. In an embodiment in which one or more, preferably two or more purified bacterial strains are used, each of the bacterial strains can have different properties or activities, e.g., produce different metabolites, produce different enzyme, confer different beneficial traits.

In certain embodiments, the bacterial population or composition may comprise bacteria of the one or more bacterial strain as described in the present specification, and optionally one or more additional bacterial strain. In certain embodiments, the bacteria of the one or more bacterial strain as described herein may collectively constitute at least about 1% by CFU of all viable bacteria constituting the bacterial population or composition, such as at least about 2%, at least about 5%, at least about 10%, preferably at least about 20%, such as at least about 30%, or at least about 40%, more preferably at least about 50%, such as at least about 60%, at least about 70%, at least about 80%, at least about 90%, at least about 95%, at least about 99%, or even 100% by CFU of all viable bacteria constituting the bacterial population or composition.

Where the bacterial population or composition comprises bacteria of two or more bacterial strains as described in the present specification, they may in certain embodiments be included in the population or composition in unequal amounts or preferably in about equal amounts. By means of an example and without limitation, the bacteria of each of the two or more bacterial strains as described herein may, each independently, constitute at least about 1% by CFU of all viable bacteria constituting the bacterial population or composition, such as at least about 2%, at least about 5%, at least about 10%, preferably at least about 20%, such as at least about 30%, or at least about 40%, more preferably at least about 50%, such as at least about 60%, at least about 70%, at least about 80%, at least about 90%, at least about 95%, or at least about 99% of all viable bacteria constituting the bacterial population or composition (where the sum of these amounts would exceed 100%, it shall be understood that the sum is capped at 100%).

Where the bacterial population or composition comprises bacteria of the one or more bacterial strain as described in the present specification and bacteria of one or more additional bacterial strain, each bacterial strain may in certain embodiments be included in the population or composition in unequal amounts or preferably in about equal amounts. By means of an example and without limitation, the bacteria of each of the bacterial strains may, each independently, constitute at least about 1% by CFU of all viable bacteria constituting the bacterial population or composition, such as at least about 2%, at least about 5%, at least about 10%, preferably at least about 20%, such as at least about 30%, or at least about 40%, more preferably at least about 50%, such as at least about 60%, at least about 70%, at least about 80%, at least about 90%, at least about 95%, or at least about 99% of all viable bacteria constituting the bacterial population or composition (where the sum of these amounts would exceed 100%, it shall be understood that the sum is capped at 100%).

In certain embodiments, the concentration or amount of each isolated bacterial strain in the bacterial population or composition may be at least about 10² CFU/ml or CFU/g, at least about 10³ CFU/ml or CFU/g, at least about 10⁴ CFU/ml or CFU/g, at least about 10⁵ CFU/ml or CFU/g, at least about 10⁶ CFU/ml or CFU/g, at least about 10⁷ CFU/ml or CFU/g, at least about 10⁸ CFU/ml or CFU/g, at least about 10⁹ CFU/ml or CFU/g, or at least about 10¹⁰ CFU/ml or CFU/g. More preferably, the concentration or amount of each isolated bacterial strain in the bacterial population or composition may be between 10³ to 10¹⁰ CFU/ml or CFU/g, between 10⁴ to 10¹⁰ CFU/ml or CFU/g, between 10⁵ to 10¹⁰ CFU/ml or CFU/g, between 10⁶ to 10¹⁰ CFU/ml or CFU/g, between 10⁶ to 10⁹ CFU/ml or CFU/g, between 10⁷ to 10⁹ CFU/ml or CFU/g, or between 10⁸ to 10⁹ CFU/ml or CFU/g.

Also provided in an aspect is a bacterial population, such as in accordance with the aforementioned explanations, comprising the bacterial strain as deposited under the Budapest Treaty at the Polish Collection of Microorganisms (PCM) under accession no. B/00365 on 11 Feb. 2022 with the following identification reference given to the deposited material by the depositor: TDC-B-A100D6.

Also provided in an aspect is an agricultural active composition, such as in accordance with the aforementioned explanations, comprising the bacterial strain as deposited under the Budapest Treaty at the Polish Collection of Microorganisms (PCM) under accession no. B/00365 on 11 Feb. 2022 with the following identification reference given to the deposited material by the depositor: TDC-B-A100D6, or a functional mutant thereof.

Bacterial strains, combinations, populations, and compositions as discussed throughout the present specification can be employed in plants cultivation, in particular to facilitate an improvement or enhancement in the biomass, number of tillers per plant, and/or yield of plants.

The terms “plant” or “plant element” as used herein encompasses whole plants, ancestors and progeny of the plants and plant parts, including seeds, shoots, stems, leaves, roots (including tubers), flowers, and tissues and organs. The terms “plant” or “plant element” also refer to plant cells, suspension cultures, callus tissue, embryos, meristematic regions, gametophytes, sporophytes, pollen and microspores. Hence, when the term “plant” or “plant element” is used herein, the term is intended to encompass “a plant, part thereof, or plant cell”. The term “plant cell” may encompass a non-propagating plant cell.

The phrases “part of a plant” or “plant part” as used herein refer to any one or more portions of a plant, such as to any one or more of the seeds, shoots, stems, leaves, roots (including tubers), flowers, and tissues and organs of a plant, such as for example meristematic tissue, ground tissue, vascular tissue, dermal tissue, etc. In addition, a “plant part” is intended to generically reference any part of a plant that is able to initiate other plants via either sexual or asexual reproduction of that plant, for example but not limited to: seed, seedling, root, shoot, cutting, scion, graft, stolon, bulb, tuber, corm, keikis, or bud.

In certain embodiments, the part of a plant may be any one or more of the seeds, shoots, stems, leaves, roots (including tubers), or flowers. In certain embodiments, the part of a plant may be the seeds. In certain embodiments, the part of a plant may be the shoots, stems, or leaves. In certain embodiments, the part of a plant may be the roots (including tubers). In certain embodiments, the part of a plant may be tissues or organs of a plant.

In certain embodiments, the seeds, shoots, stems, leaves, roots (including tubers), flowers, tissues or organs of the plant, when treated according to the methods as taught herein, may be attached to (e.g., growing on) the whole plant. In certain embodiments, the seeds, shoots, stems, leaves, roots (including tubers), flowers, tissues or organs of the plant, when treated according to the methods as taught herein, may be detached from (e.g., not growing on) the whole plant. For instance, seeds may be detached from (e.g., not growing on) the whole plant when treated according to the methods as taught herein.

In some embodiments, plants may include wild plants and domesticated varieties. In certain embodiments, plants and plant parts may be developed by any technique, including but not limited to directed evolution, selection, marker assisted selection, hybridization, outcrossing, backcrossing, in-breeding, polyploidization, reverse breeding, doubled haploids, induced mutation, other genetic or epigenetic modifications, and combinations thereof.

The phrases “locus of a plant” or “locus of growth of a plant” as used herein refers to an area in close proximity of a plant (including parts thereof such as a seed). For instance, the locus of a plant may be a circular area around the plant, e.g., around the stem of a plant or around a seed, such as a circular area having a diameter of at most 1 meter, for instance at most 50 centimeters (cm), at most 40 cm, at most 30 cm, at most 20 cm, at most 10 cm, or at most 5 cm, around the plant, e.g., around the stem of a plant or around a seed. The locus of growth may include the growth medium (e.g., soil, hydroponic medium, or hydroculture medium) for cultivating the plant.

The reference to plants includes any plants. In certain embodiments, the plants may be an angiosperm. Particularly preferred are agricultural plants. The terms “agricultural plants”, “crops” or “plants of agronomic importance” as used herein include plants that are cultivated by humans for but not limited to food, feed, fiber, fuel, gardening, and/or industrial purposes.

In certain embodiments, the plant is a monocotyledon. The terms “monocotyledon” or “monocot” refer to flowering plants (angiosperms) whose seeds typically contain only one embryonic leaf or cotyledon.

In certain preferred embodiments, the plant is a cereal plant. The terms “cereal” or “cereal plant” refer to any grass cultivated for the edible components of its grain (caryopsis), composed of the endosperm, germ, and bran.

In certain preferred embodiments, the plant is selected from the group consisting of wheat, maize, barley, rice, millet, rye, triticale, sorghum, emmer, spelt, einkorn, teff, milo, and oats. In certain particularly preferred embodiments, the plant is wheat or maize. In certain particularly preferred embodiments, the plant is wheat (Triticum aestivum and related varieties). In further particularly preferred embodiments, the plant is maize (Zea mays and related varieties).

In certain embodiments, the plant is a dicotyledon. The terms “dicotyledon” or “dicot” refer to flowering plants (angiosperms) whose seeds typically contain two embryonic leaves or cotyledons.

By means of an example and without limitation, dicot plants include tobacco, potato, sugar beet, soybean, alfalfa, brassicas, sunflower, cucurbits, peppers, tomatoes, and all other annual or perennial plants under the Dicotyledonae.

The plant may be a non-modified plant or a modified plant. As used herein, a plant may be “modified” when it comprises an artificially introduced genetic or epigenetic “modification”. In some embodiments, the modification is introduced by a genome engineering technology. In some embodiments, the modification is introduced by a targeted nuclease. In some embodiments, targeted nucleases include, but are not limited to, transcription activator-like effector nuclease (TALEN), zinc finger nuclease (ZNF), clustered regulatory interspaced short palindromic repeats (CRISPR), CRISPR/Cas9, CRISPR/CPFL and combinations thereof. In some embodiments, the modification is an epigenetic modification. In some embodiments, the modification is introduced by treatment with a DNA methyltransferase inhibitor such as 5-azacytidine, or a histone deacetylase inhibitor such as 2-amino-7-methoxy-3H-phenoxazin-3-one. In some embodiments, the modification is introduced via tissue culture. In some embodiments, a modified plant may comprise a transgene. In certain embodiments, the plant may be a non-transgenic plant or a transgenic plant. In certain preferred embodiments, the plant may be a non-transgenic plant or a transgenic plant. The terms “recombinant”, “transgenic” or “transgene” as used herein, for example with regard to a plant, refer to those plants brought about by recombinant methods in which a nucleic acid sequence and/or genetic control sequence(s) which are operably linked to the nucleic acid sequence are not located in their natural genetic environment. The natural genetic environment is understood as meaning the natural genomic or chromosomal locus in the original plant. A naturally occurring nucleic acid sequence (e.g., a naturally occurring combination of the native promoter of a nucleic acid sequence, the corresponding native nucleic acid sequence encoding a protein, and the native transcription termination sequence of a nucleic acid sequence) becomes a recombinant nucleic acid when this nucleic acid is not integrated in the natural genetic environment but in a different genetic environment as a result of an isolation of said nucleic acid from its natural genetic environment and re-insertion at a different genetic environment.

In certain embodiments, the plant may be free of disease and/or pathogen pressure and/or pest organisms. In other embodiments, the plant may be affected with disease and/or pathogen pressure and/or pest organisms.

The products, methods and uses as taught herein can provide for advantages in plants treated therewith relative to untreated plants. An “untreated plant” refers to a plant of the same species as (e.g., which is isogenic to or genetically identical to) and grown under substantially the same conditions as (e.g., for the same amount of time, in the same climate, and cultivated according to the same methods using the same materials, with biomass, yield and other characteristics being measured according to the same methods) a plant which has been administered the bacterial strain(s) (for reasons of brevity, the mention of bacterial strain(s) henceforth encompasses the one or more bacterial strain as envisaged herein, as well as the bacterial strain combinations and bacterial populations as disclosed herein, as well as the compositions comprising these, insofar the context does not indicate otherwise; these may also contain the optional further plant-beneficial microorganism(s)), except that the untreated plant has not been administered said bacterial strain(s) to the plant, a part thereof, a seed for growing the plant, or locus of the plant. The term may be used synonymously to “reference plant” or “reference”, a plant genetically identical to and handled in substantially identical ways to a treated plant, with the exception of the treatment under investigation, and which thus offers a meaningful and informative control for detecting the effects of said treatment. A treated plant and a control reference plant can thus be exposed to substantially the same environmental conditions. By means of an example, the treated plant and reference plant can both be observed under substantially identical conditions of drought stress, or the treated plant and reference plant can both be observed under substantially identical conditions of no drought stress.

In one example, two genetically identical maize plant embryos may be separated into two different groups, one receiving a treatment (such as administration of the bacterial strain(s)) and one control, e.g., reference, that does not receive such treatment. Any phenotypic differences between the two groups may thus be attributed solely to the treatment and not to any inherency of the plant's genetic makeup. In another example, two genetically identical wheat seeds may be treated with a composition, one that introduces a bacterial population and one that does not. Any phenotypic differences between the plants derived from (e.g., grown from or obtained from) those seeds may be attributed to the bacterial treatment.

The term “untreated seed” refers to a seed of the same species as (e.g., which is isogenic to or genetically identical to) and obtained under substantially the same conditions (e.g., plants from which the seeds are obtained are grown for the same amount of time, in the same climate, and cultivated according to the same methods using the same materials, seeds are stored under the same conditions) as a seed which has been administered the bacterial strain(s), except that the untreated seed has not been administered said bacterial strain(s).

The term “plant growth feature” is intended to broadly encompass any feature that relates in some way to plant growth. The feature may relate to or be observable with respect to an individual plant or to a population of plants. Examples of such features include, without limitation, plant wet or dry biomass, plant height, plant size, emergence %, emergence date, canopy cover, flowering status, seed yield, grain yield, fruit yield, number of tillers per plant, shoot length, root length, root architecture, seed weight, senescence, stay-green, number of mature plant reproductive elements per plant, visual appearance, etc.

The reference to an improvement encompasses any qualitative or quantitative change or modification in a plant growth feature that is industrially beneficial, in particular in the context of agriculture. To the extent a plant growth feature is quantifiable, an improvement may be synonymous to an increase or a reduction in that quantity, depending on the nature of the plant growth feature. By means of an example and without limitation, an increase may be desired in quantifiable features such as plant wet or dry biomass, plant height, plant size, canopy cover, seed yield, grain yield, fruit yield, number of tillers per plant, shoot length, root length, seed weight, etc.

In certain embodiments, the plant growth feature comprises or is biomass, number of tillers per plant, yield, or any combination thereof.

As used herein, the “biomass” of a plant refers to the amount (e.g., as determined by mass or weight, e.g., measured in grams of air-dry or wet tissue) or quantity (numbers) of tissue produced from the plant. Unless specified otherwise, biomass comprises both aboveground biomass (i.e., aerial biomass, including but not limited to stem, leaves, fruits, and/or seeds) and/or belowground biomass (i.e., roots). The biomass refers to the biomass at a given time. The biomass of a plant that has been administered the bacterial strain(s) can be measured according to known methods including weighing. Biomass may be given as weight per unit area. The term may also refer to all the plants or species in the community (community biomass). In certain embodiments, an increase in the biomass of a plant or part thereof may include an increase in the dry biomass, the wet biomass, the number of tillers, the height of the plant, the plant yield, the seed yield, the fruit yield, or a combination thereof.

In certain embodiments, the dry biomass of the plant may be measured according to the dry weight (DW) of the plant or part thereof in grams. The dry weight may be determined after drying the plant or part thereof until no residual water is left, e.g., after drying at 60° C. for 1 week. In certain embodiments, the wet biomass of the plant may be measured according to the wet or fresh weight of the plant or part thereof in grams. The number of tillers may be determined by counting the tillers. The height of the plant may be determined by measuring the height.

As used herein the phrase “yield” or “plant yield” refers to the amount, mass or weight (e.g., as determined by weight or size) or quantity (numbers) of tissues or organs produced per plant, per growing area, and/or per growing season.

The plant yield may be affected by various parameters including, but not limited to, plant biomass; plant vigor; growth rate; seed yield; seed or grain quantity; seed or grain quality; oil yield; content of oil, starch and/or protein in harvested organs (e.g., seeds or vegetative parts of the plant);

number of flowers (florets) per panicle (expressed as a ratio of number of filled seeds over number of primary panicles); harvest index; number of plants grown per area; number and size of harvested organs per plant and per area; number of plants per growing area (density); number of harvested organs in field; total leaf area; carbon assimilation and carbon partitioning (the distribution/allocation of carbon within the plant); resistance to shade; number of harvestable organs (e.g. seeds), seeds per pod, weight per seed; and modified architecture.

As used herein the phrases “seed yield” or “grain yield” refer to the number or weight of the seeds per plant, seeds per pod, or per growing area or to the weight of a single seed. Hence seed yield can be affected by seed dimensions (e.g., length, width, perimeter, area and/or volume), number of (filled) seeds and seed filling rate. An increased seed yield per growing area could be obtained by increasing seed yield per plant, and/or by increasing number of plants grown on the same growing area.

The term “seed” (also referred to as “grain” or “kernel”) as used herein refers to a small embryonic plant enclosed in a covering called the seed coat (usually with some stored food). The seed is the product of the ripened ovule of gymnosperm and angiosperm plants which occurs after fertilization and some growth within the mother plant. The terms “seed”, “plant seed”, or “seed for growing the pant” may be used interchangeably herein.

The biomass of a plant and/or the yield of a plant that has been administered the bacterial strain(s) can be measured at a timepoint that is between about 7 days to about 350 days, about 7 days to about 300 days, about 7 days to about 250 days, about 7 days to about 200 days, about 7 days to about 150 days, or about 10 days to about 100 days, such as about 15 days to about 75 days, about 20 days to about 60 days, or about 25 days to about 50 days following administration of said bacterial strain(s) to the plant. In certain embodiments, the biomass, number of tillers per plant, and/or yield of a plant, such as a cereal plant, that has been administered the bacterial strain(s) can be measured at the time that the plant is harvested to collect its grain or produce, i.e., at the time that the mature plant, such as a cereal plant, e.g., a wheat or maize plant, is gathered from a field. The biomass of a plant and/or the yield of a plant that has been administered the bacterial strain(s) vs. a reference plant not so treated would be measured at the same time point.

In certain embodiments, the plant growth feature may comprise dry biomass, wet biomass, number of tillers per plant, seed or grain yield, or any combination thereof. In certain embodiments, the present invention allows to improve or increase any one or more and preferably all of these features.

The term “increase” as used herein is intended to be synonymous with terms such as “upregulate”, “enhance”, “stimulate”, or “boost”. An increase in the plant biomass, number of tillers per plant, and/or yield can be in the whole plant or in any part thereof such as aboveground (harvestable) parts, vegetative biomass, roots, fruits, or seeds. Any extent or degree of such increase is contemplated herein, in particular any agronomically meaningful increase. Typically, the term may in appropriate contexts, such as in experimental or agricultural contexts, denote a statistically significant increase relative to a reference. The skilled person is able to select such a reference, as also discussed elsewhere in this specification. For example, such increase may fall outside of error margins for the reference (as expressed, for example, by standard deviation or standard error, or by a predetermined multiple thereof, e.g., ±1×SD or ±2×SD, or ±1×SE or ±2×SE). Accordingly, while the respective improvements or increases may be observable at the level of individual plants, they are more usefully evaluated by comparing relevant population characteristics, such as average or median values of the respective traits, obtained using sample sizes of treated vs. untreated plants that allow for statistically meaningful conclusions, such as for example shown in the Examples section.

In certain embodiments, the biomass, number of tillers per plant, and/or yield of a plant may each independently be increased by at least about 1% relative to (i.e., compared with) (i.e., the biomass of a plant may be at least about 1.01-fold) the biomass, number of tillers per plant, and/or yield of an untreated plant, such as preferably by at least about 2% (i.e., 1.02-fold), by at least about 3% (i.e., 1.03-fold), by at least about 5% (i.e., 1.05-fold), by at least about 10% (i.e., 1.10-fold), by at least about 15% (i.e., 1.15-fold), by at least about 20% (i.e., 1.20-fold), or more, such as by at least about 25% (i.e., 1.25-fold), by at least about 30% (i.e., 1.30-fold), by at least about 35% (i.e., 1.35-fold), by at least about 40% (i.e., 1.40-fold), by at least about 45% (i.e., 1.45-fold), by at least about 50% (i.e., 1.50-fold), or more. For example, the biomass, number of tillers per plant, and/or yield of a plant may be increased by between 2% and 5%, between 5% and 10%, between 10% and 15%, between 15% and 20%, between 20% and 30%, between 30% and 40%, or between 40% and 50% relative to the biomass, number of tillers per plant, and/or yield of an untreated plant. Such enhanced biomass, number of tillers per plant, and/or yield may advantageously reduce or even abolish the need to treat the plants with agrochemicals such as fertilizers in the field and thereby advantageously leads to more sustainable agriculture. As said above, these percentages or fold increases may conveniently reflect the relationships between averages for the respective traits in the treated vs. untreated populations, as determined by evaluating representative population samples.

In certain embodiments, the seed or grain yield of the plant may be increased, such as increased by the aforementioned percentages or fold change.

As set forth elsewhere in the specification, the bacterial strain(s) can thus be administered to the plant, a part of the plant, a seed for growing the plant, or locus of the plant in an amount effective to produce an improved plant growth feature, such as an increased biomass, number of tillers per plant, and/or yield in the plant compared to an untreated plant.

The phrase “administering” generally refers to man- and/or machine-driven or effected disposing, applying, delivering, or providing of a recited object, such as the bacterial strain(s), to a recipient entity, such as the plant, a part thereof, a seed for growing the plant, or locus of the plant. The bacterial strain(s) as taught herein may be administered by any known method wherein all or part of the plant is treated, such as by root, seed, or foliar inoculation. For example, the administration can be to the aerial portions of a plant, such as the leaves and stem, to the roots of the plant, to the seed of the plant prior to planting the seed in soil, or to the soil or plant growth medium surrounding the plant or plant seed. Application methods such as spraying, coating, covering, contacting, and/or immersion can be adopted. In certain embodiments, application may be to a surface, such as to the surface of growth medium (such as soil), plant, plant part, seeds, harvested crop, harvested seed crop, stored crops or crop parts. In certain embodiments, the administration may be to the plant, part thereof, or locus of the plant, present on the field. The terms “field” or “agricultural field” may be used interchangeably herein and refers to an area of land used for agricultural purposes such as cultivating crops, such as cultivating wheat plants or maize plants.

In certain embodiments, the bacterial strain(s) may be applied to the part of the plant, when forming part of the plant. For instance, they may be applied to the part of the plant, such as to the leaf, when the part of the plant, such as the leaf, is present on or attached to (e.g., is growing on) the plant.

In certain embodiments, the bacterial strain(s) may be applied to any one or more of the seeds, shoots, stems, leaves, roots (including tubers), flowers, tissues, or organs of a plant. In certain embodiments, the bacterial strain(s) may be applied to (e.g., sprayed on) the whole of the above-ground part of the plant. Accordingly, in certain embodiments, the bacterial strain(s) may be applied to (e.g., sprayed on) any one or more of the shoots, stems, leaves, or flowers of the plant. Preferably, the bacterial strain(s) may be applied to (e.g., sprayed on) any one or more of the shoots, leaves, or flowers of the plant. In certain embodiments, the bacterial strain(s) may be applied to (e.g., sprayed on) the shoots of the plant.

In certain embodiments, the bacterial strain(s) may be administered to the locus of the plant, such as by inoculating the growth medium. Hence, in certain embodiments, the method comprises inoculating soil or a plant growth medium with the bacteria and growing the plant in said soil or medium.

The terms “growth medium” or “plant growth medium” as used herein refer to a substrate or medium for culturing plants. In embodiments, the growth medium may be soil, compost, peat, coco-coir, wood fibers, a soil-mimicking substrate such as mineral lava or basalt substrate, textile, or a soil-less substrate. In certain embodiments, the grown medium may be sand, gravel, polysaccharide, mulch, peat moss, straw, logs, clay, or a combination thereof. In embodiments, the plant growth medium may also include a hydroculture system or an in vitro culture system. Hence, in certain embodiments, the plant growth medium is a hydroponic medium or a hydroculture medium. The skilled person understands that different types of growth media may be used for growing different types of plants. Inoculating a plant growth medium can be performed, by way of example using a liquid, a powder, a granule, a pellet.

Hydroculture, also encompassing hydroponics, is the growing of plants in a soil-less medium or an aquatic based environment, while in vitro culture system refers to the growing of plants or explants on or in a recipient with synthetic medium, in sterile conditions, in a controlled environment and in reduced space. Explants refer to parts of a plant, from all the aerial part to isolated cells, as parts of leaves, of roots, seeds, bulbs, tubers, buds. The inoculation of the plant growth medium with the bacterial strain(s) may be performed before, during and/or after sowing or before, during and/or after the start of the plant growth cycle in case of hydroculture or in vitro culture. The inoculation can be performed once or multiple times during the plant growth cycle.

In certain embodiments, sprayable liquids may be applied by spraying the plant, part thereof, or locus of the plant by conventional spraying equipment as known in the art, such as airplanes, backpack sprayers, tractor mounted boom sprayers etc.

In certain embodiments, application of the bacterial strain(s) to the plant, part thereof, or locus of growth of the plant may be carried out directly or by action on their surroundings or habitat using customary treatment methods, for example by dipping, drenching, spraying, coating, atomizing, irrigating, evaporating, dusting, fogging, broadcasting, foaming, painting, spreading-on, watering (drenching) or drip irrigating. In embodiments, the method may comprise spraying, sprinkling, showering, spritzing, spreading in droplets, spattering; dispersing, diffusing, or douching the plant, part thereof, or locus of growth of the plant with the bacterial strain(s).

In certain embodiments, the bacteria of the one or more bacterial strain as taught herein may be applied to a locus where plants are or are to be grown, such as upon soil, such as upon a field or within a greenhouse, in an amount of from about 1×10⁸ CFU/hectare to about 1×10¹⁴ CFU/ha, such as from about 1×10⁹ CFU/hectare to about 1×10¹³ CFU/ha, or from about 1×10¹⁰ CFU/hectare to about 1×10¹² CFU/ha, preferably about 1×10¹¹ CFU/hectare.

In certain embodiments, the application may be one-time (single) administration, repeated administration (i.e., more than one time administration) at the same or varying time intervals, or continuous administration. The bacterial strain(s) can be administered at any point in the life cycle of the plant (e.g., before or after germination). For example, administration can be to a plant's seed prior to planting the seed in soil and prior to germination. Alternatively, administration can be to the plant (e.g. a seedling), the seed of the plant, or the soil surrounding the plant after germination has occurred. Once treated with the bacterial strain(s), seeds can be planted in soil and cultivated using conventional methods for generating plant growth.

In certain embodiments, the bacterial strain(s) may be applied at a temperature (e.g., air temperature) in the range from −1° C. to 30° C. In embodiments, the application may be at a temperature in the range from 0° C. to 30° C., from 1° C. to 30° C., from 5° C. to 25° C., or from 10° C. to 20° C.

In certain embodiments, the bacterial strain(s) may be applied to the part of the plant, when not forming part of the plant. For instance, they may be applied to the part of the plant, such as to a seed for growing the plant, when the part of the plant, such as the seed, is not present on or is detached from (e.g., is not growing on) the plant. For instance, they may be applied to seeds after they have been harvested from (e.g. mechanically or manually separated from) the plant. In certain embodiments, the method may comprise administering the bacterial strain(s) to a seed of the plant, e.g., prior to planting the seed or with the seed at planting. Hence, in certain embodiments, the method comprises administering the bacterial strain(s) to a seed of the plant. In certain embodiments, the bacterial strain(s) are administered to the seed of the plant prior to planting the seed, or with the seed at planting, or after planting the seed and before germination of the seed.

In certain embodiments, the purified bacterial strain(s) are capable of colonizing plants. Successful colonization can be confirmed by detecting the presence of the strain within the plant. For example, after applying the strain to the plant parts, high titers of the strain can be detected in the roots and shoots of the plants that germinate from said plant parts such as seeds. Detecting the presence of the strain inside the plant can be accomplished by measuring the viability of the strain after surface sterilization of the plant element or the plant: strain colonization results in an internal localization of the strain, rendering it resistant to conditions of surface sterilization. The presence and quantity of strain can also be established using other means known in the art, for example, immunofluorescence microscopy using microbe-specific antibodies, or fluorescence in situ hybridization. Alternatively, specific nucleic acid probes recognizing conserved sequences from an strain can be employed to amplify a region, for example by quantitative PCR, and correlated to CFUs by means of a standard curve.

Hence, in certain embodiments, microorganisms such as bacteria are said to colonize a plant, plant part, root or seed, when they can exist in relationship with a plant or plant part during at least part of either the plant's or the microorganism's life cycle. In certain embodiments, microorganisms such as bacteria are said to colonize a plant when they can be stably detected within the plant or plant part over a period time, such as one or more days, weeks, months or years. The compositions and methods described herein may comprise one or a plurality of bacterial strains as taught herein and optionally one or more further plant-beneficial microorganism in amounts effective to colonize a plant.

In embodiments, the strains described herein may be capable of moving from one tissue type to another. For example, the detection and isolation of strains within the mature tissues of plants after treating the exterior of a plant part demonstrates their ability to move from the plant part into the vegetative tissues of a maturing plant. Therefore, in some embodiments, the population of bacterial strains is capable of moving from the plant element exterior into the vegetative tissues of a plant. In some embodiments, the strain that is disposed onto the plant element of a plant is capable, upon germination of the plant part into a vegetative state, of localizing to a different tissue of the plant.

For example, strains can be capable of localizing to any one of the tissues in the plant, including: the root, adventitious root, seminal root, root hair, shoot, leaf, flower, ear, spike, spikelet, bud, tassel, meristem, pollen, pistil, ovaries, stamen, fruit, stolon, rhizome, nodule, tuber, trichome, guard cells, hydathode, petal, sepal, glume, rachis, vascular cambium, phloem, and xylem. In an embodiment, the strain is capable of localizing to the root and/or the root hair of the plant. In another embodiment, the strain is capable of localizing to the photosynthetic tissues, for example, leaves and shoots of the plant. In other cases, the strain is localized to the vascular tissues of the plant, for example, in the xylem and phloem. In still another embodiment, the strain is capable of localizing to the reproductive tissues (flower, pollen, pistil, ovaries, stamen, fruit, spike, spikelet) of the plant. In another embodiment, the strain is capable of localizing to the root, shoots, leaves and reproductive tissues of the plant. In still another embodiment, the strain colonizes a fruit or plant element tissue of the plant. In still another embodiment, the strain is able to colonize the plant such that it is present in the surface of the plant (i.e. its presence is detectably present on the plant exterior). In still other embodiments, the strain is capable of localizing to substantially all, or all, tissues of the plant. In some cases, strains are capable of replicating within the host plant and colonizing the plant.

In further embodiments, following administration of the bacterial strain(s), the plants are cultivated under conditions to promote plant growth and development. In other words, the present methods may further comprise cultivating the plant under conditions to promote plant growth and development.

In certain embodiments, the method comprises administering the bacterial strain(s) to a seed of the plant, a whole plant, or a seedling, and optionally cultivating the seed, whole plant, or seedling under conditions to promote plant growth and development. In certain embodiments, the method comprises administering the bacterial strain(s) to a seed of the plant, and optionally cultivating the seed under conditions to promote plant growth and development. Hence, in such latter embodiments, the plant is grown from a seed, in particular a seed planted in said soil or plant growth medium.

Cultivating the seed under conditions promoting plant growth and development, may but need not include growth to maturity and/or regeneration.

In certain embodiments, the method may comprise further propagating the plant treated with the bacterial strain(s). Accordingly, the invention provides a method for increasing biomass, number of tillers per plant, and/or yield of a plant grown from a seed compared to a plant grown from an untreated seed, the method comprising administering the bacterial strain(s) to the seed for growing the plant. The invention also provides the use of the bacterial strain(s) for increasing biomass, number of tillers per plant, and/or yield of a plant grown from a seed compared to a plant grown from an untreated seed.

Also provided herein is a method of treating a seed of a plant comprising inoculating the seed with bacteria of one or more bacterial strain as taught herein, such that the bacteria colonize a plant germinated from the inoculated seed and/or the soil or plant growth medium surrounding the growing plant, whereby a plant growth feature of the plant, such as the biomass, number of tillers per plant, and/or yield of the plant, is improved compared to a plant germinated from an untreated seed. In certain embodiments, the seed is coated with the bacteria, incubated with the bacteria, or planted near the bacteria. In certain embodiments, the seed is further inoculated with one or more additional plant-beneficial microorganism as taught herein.

In certain embodiments, the bacteria of one or more bacterial strain as taught herein may be contacted with seeds in an amount of between about 1×10⁶ CFU/kg seeds to about 1×10¹² CFU/kg seed, such as between about 1×10⁷ CFU/kg seeds to about 1×10¹¹ CFU/kg seeds, or between about 1×10⁸ CFU/kg seeds to about 1×10¹⁰ CFU/kg seeds, or between about 1×10⁹ CFU/kg seeds to about 1×10¹⁰ CFU/kg seeds, preferably about 2.5×10⁹ CFU/kg. In certain embodiments, seeds may be treated with cultivated bacteria of one or more bacterial strain as taught herein, such as with a bacterial sample or culture showing Optical Density (OD) between about 0.1 and about 1 at wavelength 600 nm, at an amount of between about 0.1 l to about 5 l of the culture per kg of seeds, such as about 4.6 l.

In certain embodiments, the bacteria of one or more bacterial strain as taught herein may be coated on or present on or inoculated into seeds at a quantity of, on average, at least 10 CFU per seed, preferably at least 100 CFU per seed, more preferably at least 500 CFU per seed, and even more preferably at least 1000 CFU per seed, such as between 1×10³ and 1×10⁵ CFU per seed, or between 1×10³ and 1×10⁴ CFU per seed. The phrase “CFU per seed”, herein and throughout the specification, also encompasses the reference to “spores per seed”, in case the microorganism lends itself to being administered in the form of spores. Certain embodiments thus provide a plant seed comprising at least 10 CFU, preferably at least 100 CFU, more preferably at least 500 CFU, and even more preferably at least 1000 CFU, such as between 1×10³ and 1×10⁵ CFU, or between 1×10³ and 1×10⁴ CFU, of the bacteria as taught herein. For example, the seed may be coated with the bacterial cells or the composition as taught herein.

In certain embodiments, the bacteria of one or more bacterial strain as taught herein can be cultured on a culture medium or can be adapted to culture on the culture medium. Said culture medium is sterile prior to being inoculated with the bacterial strain and comprises all nutrients for growth and maintenance of the strain on the culture medium. In addition, the culture medium can be in a solid, semi-solid or liquid form.

In certain embodiments, the bacteria of one or more bacterial strain as taught herein may be administered as a bacterial sample, culture, or inoculum (e.g., showing Optical Density (OD) between about 0.1 and about 1 at wavelength 600 nm, for example measured using an Implen GmbH (Munich, Germany) spectrophotometer, spray dried bacteria, freeze dried bacteria, suspension of spray dried or freeze dried bacteria, etc.

In certain embodiments, the bacteria of one or more bacterial strain as taught herein may thus be administered as a whole cell broth, comprising the bacteria as well as the medium in which the bacteria have been grown.

A further aspect provides a plant or part thereof treated with the bacterial strain(s). Also provided is a plant or part thereof heterologously disposed with the bacteria as taught herein. In certain embodiments, the plant part may be a seed, such as a seed coated with the bacteria or the composition. The plant or part thereof or a plant grown from said plant part can display increased biomass as compared to an untreated plant or part thereof.

Hence, also disclosed is a plant seed, preferably a crop plant seed (e.g., the seed of a wheat plant or maize plant), treated with, such as coated with, the bacterial strain(s), e.g., such that all or part of the seed has a coating or film comprising the bacteria. The plant grown from the treated seed can display increased biomass as compared to a plant grown from an untreated seed.

Further provided is a plant grown from a plant or part thereof (e.g., seed) treated with the bacterial strain(s).

In an embodiment, bacteria of the one or more strain as taught herein may, collectively or preferably each of the strains individually and independently, be present in an amount of at least about 10² CFU per treated plant or part thereof. In an embodiment, bacteria of the one or more strain as taught herein may, collectively or preferably each of the strains individually and independently, be present in an amount of at least about 10² CFU per plant grown from the treated plant or part thereof such as from a treated seed.

Preferably, bacteria of the one or more strain as taught herein may, collectively or preferably each of the strains individually and independently, be present on the plant or part thereof in an amount effective to be detectable within a target tissue of the mature plant selected from a fruit, a seed, a leaf, or a root, or portion thereof. For example, in an amount of at least about 100 CFU, between 100 and 200 CFU, at least about 200 CFU, between 200 and 300 CFU, at least about 300 CFU, between 300 and 400 CFU, at least about 500 CFU, between 500 and 1,000 CFU, at least about 1,000 CFU, between 1,000 and 3,000 CFU, at least about 3,000 CFU, between 3,000 and 10,000 CFU, at least about 10,000 CFU, between 10,000 and 30,000 CFU, at least about 30,000 CFU, between 30,000 and 100,000 CFU, at least about 10⁵ CFU, between 10⁵ and 10⁶ CFU, at least about 10⁶ CFU or more in the mature plant.

In certain embodiments, the plant or part thereof, such as a seed, treated with (e.g. coated with) the bacteria as taught herein may be shelf-stable. The bacterial strain may be shelf-stable, where at least 0.01%, of the CFUs are viable after storage in desiccated form (i.e., moisture content of 30% or less) for 1, 2, 3, 4, 5, 6, 7, 8, 9, 10 or greater than 10 weeks at 4° C. or at room temperature. Optionally, a shelf-stable composition comprising the bacteria may be in a dry composition, a powder composition, or a lyophilized composition. In an embodiment, the composition may be formulated to provide stability for the strains. In an embodiment, the plant or part thereof, such as a seed, treated with (e.g. coated with) the bacteria may be substantially stable at temperatures between about −20° C. and about 50° C. for at least about 1, 2, 3, 4, 5, or 6 days, or 1, 2, 3 or 4 weeks, or 1, 2, 3, 4, 5, 6, 7, 8, 9, 10, 11 or 12 months, or one or more years. In another embodiment, the plant or part thereof, such as a seed, treated with (e.g. coated with) the bacteria may be substantially stable at temperatures between about 4° C. and about 37° C. for at least about 5, 10, 15, 20, 25, 30 or greater than 30 days. Preferably the plant or part thereof, such as a seed, treated with (e.g. coated with) the bacteria is substantially stable at temperatures between about 4° C. and about 37° C. for at least one year or greater than one year.

In certain embodiments, the plant or part thereof, such as a seed, treated with (e.g. coated with) the bacteria are confined within a suitable container, such as an object selected from the group consisting of: bottle, jar, ampule, package, vessel, bag, box, bin, envelope, carton, container, silo, shipping container, truck bed, and case.

In certain embodiments, the bacteria as taught herein are heterologous to the plant or plant part to be treated or administered with the same. A bacterium is considered heterologous to the plant, plant part, seed for growing the plant, or locus of the plant if the plant, plant part, seed for growing the plant, or locus of the plant that is untreated (e.g., a seed that is not treated with a bacterial strain described herein) does not contain detectable levels of the bacterium. A bacterium is considered “heterologously disposed” or “heterologous disposed” on the exterior surface of or within a plant or plant tissue when the bacterium is applied or disposed on the plant in a number that is not found on that plant before application of the bacterium. For example, a purified bacterial strain disposed on an exterior surface or within the seed can be an endophytic bacterium that may be associated with the mature plant, but is not found on the surface of or within the seed. As such, a bacterium is deemed heterologously disposed when applied on the plant that either does not naturally have the bacterium on its surface or within the particular tissue to which the bacterium is disposed, or does not naturally have the bacterium on its surface or within the particular tissue in the number that is being applied.

In certain embodiments, the strain is heterologous disposed, for example, on the surface of a reproductive element of a plant, in an amount effective to be detectable in the mature plant. In a particular embodiment, the strain is heterologous disposed in an amount effective to be detectable in an amount of at least about 100 CFU, between 100 and 200 CFU, at least about 200 CFU, between 200 and 300 CFU, at least about 300 CFU, between 300 and 400 CFU, at least about 500 CFU, between 500 and 1,000 CFU, at least about 1,000 CFU, between 1,000 and 3,000 CFU, at least about 3,000 CFU, between 3,000 and 10,000 CFU, at least about 10,000 CFU, between 10,000 and 30,000 CFU, at least about 30,000 CFU, between 30,000 and 100,000 CFU, at least about 100,000 CFU or more in the mature plant.

In a preferred embodiment, the bacteria are heterologously disposed to a plant, part thereof, seed for growing the plant, or locus of the plant in an amount effective to improve the plant growth feature, such as increase the biomass, number of tillers per plant, and/or yield, of the treated plant relative to an untreated plant. In a preferred embodiment, the amount of the heterologous disposed strain to the plant, part thereof, seed for growing the plant, or locus of the plant is effective to maintain a critical population mass in the plant. In a further embodiment, the amount of the heterologous disposed strain to a seed for growing a plant is effective to maintain a critical population mass in the mature plant germinated from the seed. Hence, in certain embodiments, any of the bacterial strains, bacterial populations, or microbial active ingredients as contemplated herein may be heterologous disposed to the plant, plant part, or seed.

The present application also provides aspects and embodiments as set forth in the following Statements. In these statements, the wording “The [subject] according to Statement [number], wherein . . . ” or “The [subject] according to any one of Statements [numbers], wherein . . . ” also discloses and may be replaced by the simple wording “In certain embodiments . . . ”.

Statement 1. A method for improving a plant growth feature of a plant compared to an untreated plant, the method comprising administering bacteria of a bacterial strain which comprises a 16S polynucleotide having at least 99.34% sequence identity to SEQ ID NO: 1 to the plant, a part thereof, a seed for growing the plant, or a locus of the plant.

Statement 2. The method according to Statement 1, wherein the plant growth feature comprises or is biomass, number of tillers per plant, yield, or any combination thereof.

Statement 3. The method according to Statement 2, wherein the biomass of the plant is increased by at least about 2%, or by at least about 3%, or by at least about 5%, or by at least about 10%, or by at least about 15%, or by at least about 20%.

Statement 4. The method according to Statement 2 or 3, wherein the number of tillers per plant is increased by at least about 2%, or by at least about 3%, or by at least about 5%, or by at least about 10%, or by at least about 15%, or by at least about 20%.

Statement 5. The method according to any one of Statements 2 to 4, wherein the yield of the plant is increased by at least about 2%, or by at least about 3%, or by at least about 5%, or by at least about 10%, or by at least about 15%, or by at least about 20%.

Statement 6. The method according to any one of Statements 1 to 5, wherein the bacterial strain comprises a 16S polynucleotide having at least 99.41% sequence identity to SEQ ID NO: 1.

Statement 7. The method according to any one of Statements 1 to 5, wherein the bacterial strain comprises a 16S polynucleotide having at least 99.48% sequence identity to SEQ ID NO: 1.

Statement 8. The method according to any one of Statements 1 to 5, wherein the bacterial strain comprises a 16S polynucleotide having at least 99.54% sequence identity to SEQ ID NO: 1.

Statement 9. The method according to any one of Statements 1 to 5, wherein the bacterial strain comprises a 16S polynucleotide having at least 99.61% sequence identity to SEQ ID NO: 1.

Statement 10. The method according to any one of Statements 1 to 5, wherein the bacterial strain comprises a 16S polynucleotide having at least 99.67% sequence identity to SEQ ID NO: 1.

Statement 11. The method according to any one of Statements 1 to 5, wherein the bacterial strain comprises a 16S polynucleotide having at least 99.74% sequence identity to SEQ ID NO: 1.

Statement 12. The method according to any one of Statements 1 to 5, wherein the bacterial strain comprises a 16S polynucleotide having at least 99.80% sequence identity to SEQ ID NO: 1.

Statement 13. The method according to any one of Statements 1 to 5, wherein the bacterial strain comprises a 16S polynucleotide having at least 99.87% sequence identity to SEQ ID NO: 1.

Statement 14. The method according to any one of Statements 1 to 5, wherein the bacterial strain comprises a 16S polynucleotide having at least 99.93% sequence identity to SEQ ID NO: 1.

Statement 15. The method according to any one of Statements 1 to 5, wherein the bacterial strain comprises a 16S polynucleotide having 100.00% sequence identity to SEQ ID NO: 1.

Statement 16. The method according to any one of Statements 1 to 5, wherein the bacterial strain comprises a 16S polynucleotide as set forth in SEQ ID NO: 1.

Statement 17. The method according to any one of Statements 1 to 16, wherein the bacterial strain is a Streptomyces tendae or a Streptomyces marokkonensis strain.

Statement 18. The method according to any one of Statements 1 to 17, wherein the bacterial strain is the strain as deposited under the Budapest Treaty at the Polish Collection of Microorganisms (PCM) under accession no. B/00365 on 11 Feb. 2022 with the following identification reference given to the deposited material by the depositor: TDC-B-A100D6.

Statement 19. The method according to any one of Statements 1 to 17, wherein the bacterial strain is a functional mutant of the strain as deposited under the Budapest Treaty at the Polish Collection of Microorganisms (PCM) under accession no. B/00365 on 11 Feb. 2022 with the following identification reference given to the deposited material by the depositor: TDC-B-A100D6.

Statement 20. The method according to any one of Statements claims 1 to 19, comprising administering to the plant, the part thereof, the seed for growing the plant, or the locus of the plant bacteria of two or more bacterial strains, each independently as defined in any one of Statements 1 and 6-19.

Statement 21. The method according to any one of Statements claims 1 to 20, comprising administering to the plant, the part thereof, the seed for growing the plant, or the locus of the plant the bacteria in conjunction with one or more additional plant-beneficial microorganism.

Statement 22. The method according to any one of Statements 1 to 21, wherein the bacteria are administered in an agricultural active composition.

Statement 23. The method according to Statement 22, wherein the composition further comprises one or more agriculturally acceptable auxiliary.

Statement 24. The method according to Statement 23, wherein the agriculturally acceptable auxiliary is selected from a group consisting of a solvent, a carrier, a surfactant, a sticker, an antifreeze agent, a thickener, a buffering agent, an antifoaming agent, an antioxidant, a preservative, an aroma, a colorant, and combinations thereof.

Statement 25. The method according to any one of Statements 22 to 24, wherein the composition is a liquid composition.

Statement 26. The method according to Statement 25, wherein the composition is an aqueous composition.

Statement 27. The method according to Statement 25 or 26, wherein the composition is a sprayable liquid or a concentrate.

Statement 28. The method according to any one of Statements 25 to 27, wherein the composition comprises the bacteria at a concentration of at least about 10² CFU/ml; the reference to CFU/ml also encompasses spores/ml.

Statement 29. The method according to Statement 28, wherein the composition comprises two or more bacterial strains, each independently as defined in any one of Statements 1 and 6-19, and the composition comprises at least about 10² CFU/ml of bacteria of each of the strains individually, or at least about 10² CFU/ml of bacteria of all the strains collectively.

Statement 30. The method according to any one of Statements 22 to 24, wherein the composition is a non-liquid composition.

Statement 31. The method according to Statement 30, wherein the composition is a powder.

Statement 32. The method according to Statement 30 or 31, wherein the composition comprises the bacteria at an amount of at least about 10² CFU/g; the reference to CFU/g also encompasses spores/g.

Statement 33. The method according to Statement 32, wherein the composition comprises two or more bacterial strains, each independently as defined in any one of Statements 1 and 6-19, and the composition comprises at least about 10² CFU/g of bacteria of each of the strains individually, or at least about 10² CFU/g of bacteria of all the strains collectively.

Statement 34. The method according to any one of Statements 1 to 33, wherein the method comprises administering the bacteria or the composition to a seed of the plant, a whole plant, or a seedling.

Statement 35. The method according to Statement 34, wherein the bacteria or the composition are administered to the seed of the plant, such as prior to planting the seed, or with the seed at planting, or after planting the seed and before germination of the seed.

Statement 36. The method according to Statement 34 or 35, further comprising cultivating the seed, whole plant, or seedling under conditions to promote plant growth and development.

Statement 37. The method according to any one of Statements 1 to 36, wherein the method comprises inoculating soil or a plant growth medium with the bacteria and growing the plant in said soil or medium.

Statement 38. The method according to Statement 37, wherein the plant growth medium is a hydroponic medium or a hydroculture medium.

Statement 39. The method according to Statement 37 or 38, wherein the plant is grown from a seed, in particular a seed planted in said soil or plant growth medium.

Statement 40. A method of treating a seed of a plant comprising inoculating the seed with bacteria of one or more bacterial strain as defined in any one of Statements 1 and 6-19, such that the bacteria colonize a plant germinated from the inoculated seed and/or the soil or plant growth medium surrounding the growing plant, whereby a plant growth feature of the plant is improved compared to a plant germinated from an untreated seed.

Statement 41. The method according to Statement 40, wherein the plant growth feature comprises or is biomass, number of tillers per plant, yield, or any combination thereof.

Statement 42. The method according to Statement 41, wherein the biomass of the plant is increased by at least about 2%, or by at least about 3%, or by at least about 5%, or by at least about 10%, or by at least about 15%, or by at least about 20%.

Statement 43. The method according to Statement 41 or 42, wherein the number of tillers per plant is increased by at least about 2%, or by at least about 3%, or by at least about 5%, or by at least about 10%, or by at least about 15%, or by at least about 20%.

Statement 44. The method according to any one of Statements 41 to 43, wherein the yield of the plant is increased by at least about 2%, or by at least about 3%, or by at least about 5%, or by at least about 10%, or by at least about 15%, or by at least about 20%.

Statement 45. The method according to any one of Statements 40 to 44, wherein the seed is coated with the bacteria, incubated with the bacteria, or planted near the bacteria.

Statement 46. The method according to any one of Statements 40 to 45, wherein the seed is further inoculated with one or more additional plant-beneficial microorganism.

Statement 47. A plant or part thereof treated with the bacteria as defined in any one of Statements 1 and 6-19 or with the composition as defined in any one of Statements 22-33; or a plant or plant part grown therefrom.

Statement 48. A plant or part thereof heterologously disposed with the bacteria as defined in any one of Statements 1 and 6-19; or a plant or plant part grown therefrom.

Statement 49. The plant or part thereof according to Statement 47 or 48, wherein the plant part is a seed.

Statement 50. The seed according to claim 49, which is coated with the bacteria or the composition.

Statement 51. A plant seed comprising at least 10 CFU of the bacteria as defined in any one of Statements 1 and 6-19.

Statement 52. A method comprising growing a plant from the seed as defined in Statement 51 and harvesting the plant or part of the plant, such as harvesting the seeds or grains of the plant.

Statement 53. A bacterial strain as deposited under the Budapest Treaty at the Polish Collection of Microorganisms (PCM) under accession no. B/00365 on 11 Feb. 2022 with the following identification reference given to the deposited material by the depositor: TDC-B-A100D6, or a functional mutant thereof.

Statement 54. A bacterial population comprising the strain as defined in Statement 53.

Statement 55. An agricultural active composition comprising the strain as defined in Statement 53.

Statement 57. The composition according to Statement 55, further comprising one or more agriculturally acceptable auxiliary.

Statement 57. The composition according to Statement 56, wherein the agriculturally acceptable auxiliary is selected from a group consisting of a solvent, a carrier, a surfactant, a sticker, an antifreeze agent, a thickener, a buffering agent, an antifoaming agent, an antioxidant, a preservative, an aroma, a colorant, and combinations thereof.

Statement 58. The composition according to any one of Statements 55 to 57, wherein the composition is a liquid composition.

Statement 59. The composition according to Statement 58, wherein the composition is an aqueous composition.

Statement 60. The composition according to Statement 58 or 59, wherein the composition is a sprayable liquid or a concentrate.

Statement 60. The composition according to any one of Statements 58 to 60, wherein the composition comprises the bacteria at a concentration of at least about 10² CFU/ml; the reference to CFU/ml also encompasses spores/ml.

Statement 62. The composition according to Statement 61, wherein the composition comprises two or more bacterial strains, each independently as defined in any one of Statements 1 and 6-19, and the composition comprises at least about 10² CFU/ml of bacteria of each of the strains individually, or at least about 10² CFU/ml of bacteria of all the strains collectively.

Statement 63. The composition according to any one of Statements 55 or 56, wherein the composition is a non-liquid composition.

Statement 64. The composition according to Statement 63, wherein the composition is a powder.

Statement 65. The composition according to Statement 63 or 64, wherein the composition comprises the bacteria at an amount of at least about 10² CFU/g; the reference to CFU/g also encompasses spores/g.

Statement 66. The composition according to Statement 65, wherein the composition comprises two or more bacterial strains, each independently as defined in any one of Statements 1 and 6-19, and the composition comprises at least about 10² CFU/g of bacteria of each of the strains individually, or at least about 10² CFU/g of bacteria of all the strains collectively.

Statement 67. A method of genetically modifying the bacterial strain as deposited under the Budapest Treaty at the Polish Collection of Microorganisms (PCM) under accession no. B/00365 on 11 Feb. 2022, such as by mutagenesis and/or by transgenesis, for example by the introduction of one or more transgenes thereto, whereby a functional mutant of the strain is obtained.

Statement 68. The method according to any one of Statements 1 to 45, wherein the plant is a monocotyledon or a dicotyledon, preferably monocotyledon.

Statement 69. The method according to any one of Statements 1 to 45, wherein the plant is a cereal.

Statement 70. The method according to any one of Statements 1 to 45, wherein the plant is selected from the group consisting of wheat, maize, barley, rice, millet, rye, triticale, sorghum, emmer, spelt, einkorn, teff, milo, and oats.

Statement 71. The method according to any one of Statements 1 to 45, wherein the plant is wheat or maize.

Statement 72. The method according to any one of Statements 1 to 45, wherein the plant is wheat.

Statement 73. The plant or plant part according to any one of Statements 47 to 50, wherein the plant is a monocotyledon or a dicotyledon, preferably monocotyledon.

Statement 74. The plant or plant part according to any one of Statements 47 to 50, wherein the plant is a cereal.

Statement 75. The plant or plant part according to any one of Statements 47 to 50, wherein the plant is selected from the group consisting of wheat, maize, barley, rice, millet, rye, triticale, sorghum, emmer, spelt, einkorn, teff, milo, and oats.

Statement 76. The plant or plant part according to any one of Statements 47 to 50, wherein the plant is wheat or maize.

Statement 77. The plant or plant part according to any one of Statements 47 to 50, wherein the plant is wheat.

The above aspects and embodiments are further supported by the following non-limiting examples.

### EXAMPLES

### Example 1: Storage, Cultivation, and Formulation of Bacteria According to Certain Embodiments of the Invention

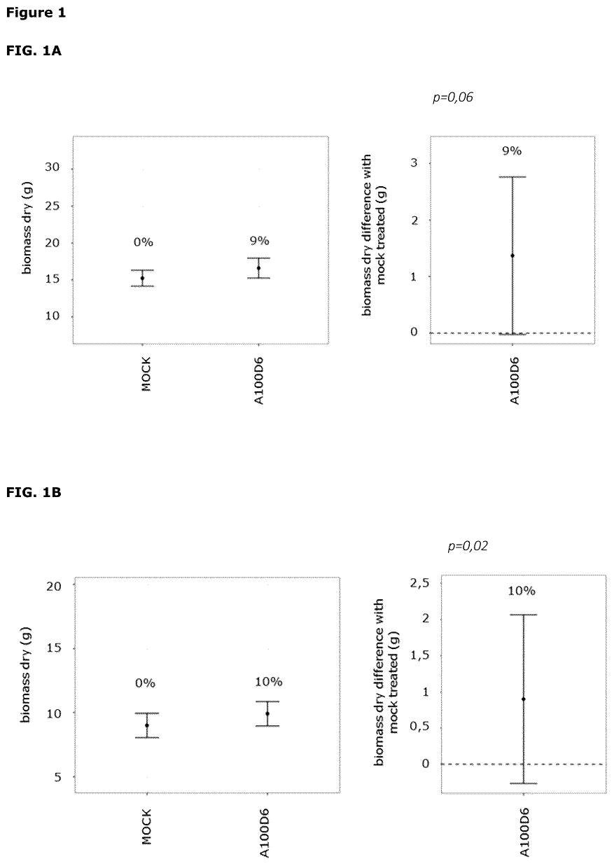

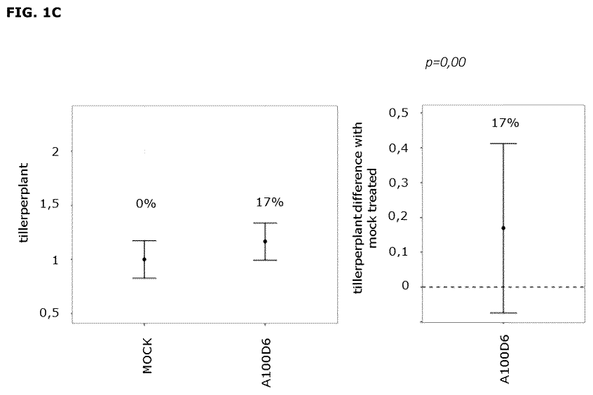

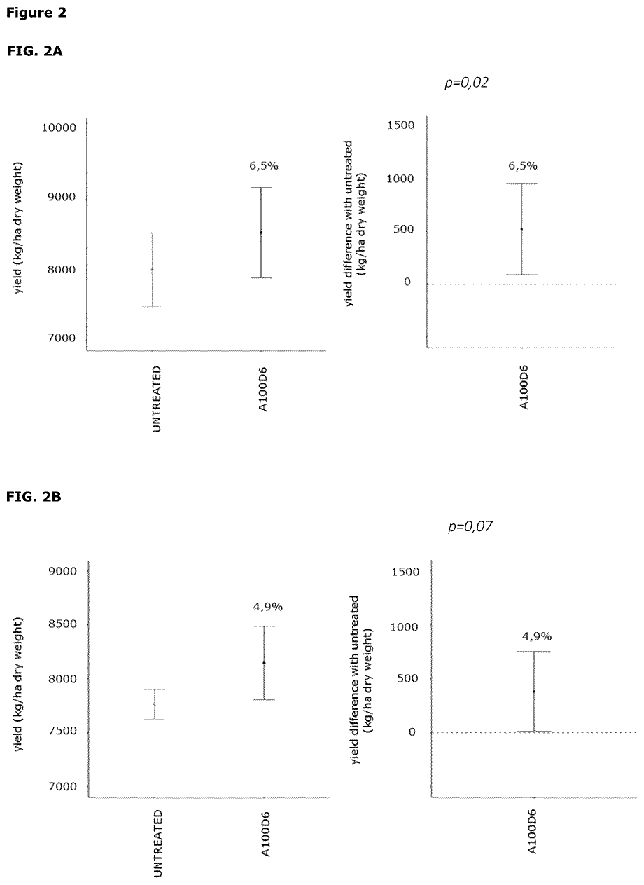

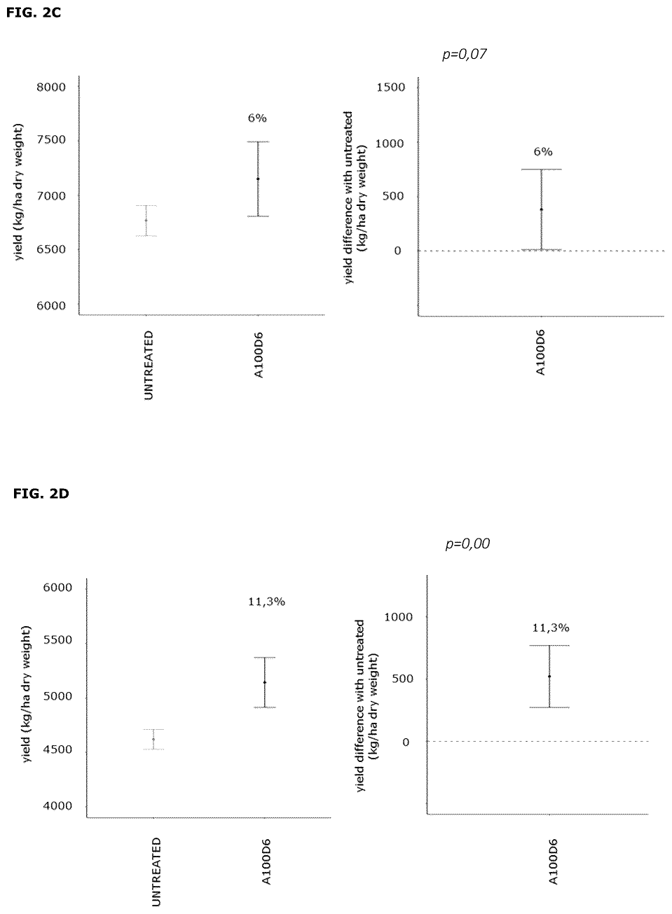

The bacterial strain deposited at the Polish Collection of Microorganisms (PCM) on 11 Feb. 2022 under accession no. B/00365 with the following identification reference given to the deposited material by the depositor: TDC-B-A100D6 (the inventors originally proposed the taxonomic designation Streptomyces tendae for the strain, whereas subsequent characterisation suggests that the strain may be more suitably classified as a Streptomyces marokkonensis strain) (henceforth referred to in the Examples as “the A100D6 strain”, “the bacterial strain”, or “the strain”) was stored at −75° C. in nutrient broth (yeast extract 1.5% w/v, peptone 2.5% w/v, 2.5% w/v sodium chloride, in distilled water, pH 6.8±0.2) amended with 25% v/v glycerol and was cultured on nutrient agar (composition as nutrient broth with 1.5% w/v agar) for at least three days at 28° C. Cell suspensions (as used for instance in the experiments shown in FIG. 1 B-C) were prepared by harvesting cells which have been incubated from single colonies in nutrient broth between two and five days in a shaker at 28° C. degrees. Harvesting was done through centrifugation at 5000 g for 5 minutes. Alternatively, the whole cell broth, comprising the bacteria as well as the medium in which the bacteria can be used for inoculation. Bacterial biomass was resuspended in 1500 microliter 1% Carboxymethycelluose (w/v, dissolved in phosphate buffer) and added to 7.2 gram of wheat seeds for greenhouse (GH) testing (see Example 2). To produce spores (as used for instance in the experiments shown in FIG. 1A and FIG. 2A-D), 400 microliter cells which have been incubated from single colonies in nutrient between two and five days in a shaker at 28° C. degrees were used to inoculate soil extract agar (20% w/v soil, in distilled water, pH 7±0.2, 2% w/v agar) at 25° C. degrees until sporulation. Spores were harvested from the plates and amended with 25% v/v glycerol stored at −75° C. Bacterial spores were resuspended in 1500 microliter 1% Carboxymethycelluose (w/v, dissolved in phosphate buffer) and added to 7.2 gram of wheat seeds for greenhouse (GH) testing (see Example 2).

For field trial testing, per kg of seeds to be coated, 40 ml of a sticker solution in water containing 1% w/v methyl cellulose and 1% w/v colorant (Agrocer® Red 112, Clariant International, Muttenz, Switzerland) together with the bacterial spores was added to the seeds. The final CFU per seed for the bacterial strain A100D6 was at least 1×10\hat{ }2 spores per seed. Seeds were stored at 4° C. before sowing (see Example 3).

### Example 2: Increased Dry Biomass and Tillers Per Plant in Wheat Treated by a Method According to Certain Embodiments of the Invention

Per treatment, 5×24 wheat seeds were treated with a formulation containing the Streptomyces strain A100D6, as described in Example 1. Five planter boxes were filled with potting soil mix and saturated with water. As a control, 10×24 wheat seeds were treated with a formulation without bacterial strain to compare (mock treatment). Seeds were sown in three rows of 8 seeds per planter box. Nutrients were being added to the planter boxes at two and three weeks after sowing. The number of tillers per plant were counted 5 weeks after sowing from the wheat plants obtained from seeds treated with said bacterial strain. After 6 weeks of growth, shoots were cut off and fresh biomass (in g) was weighed per planter box (i.e., all 24 shoots together). Plant shoots were then dried at 60° C. for 1 week and dry biomass (in g) was determined per planter box.

For all evaluated formulations, each containing a bacterial strain according to an embodiment of the invention, an increase in dry biomass and/or an increase of the number of tillers per plant was seen compared to the formulation without bacterial strain. The results of wheat treated with a formulation containing the Streptomyces strain A100D6 are shown in FIG. 1.

The graphs visualize the values of different parameters with 95% confidence intervals for A100D6 treated seeds and mock treated seeds. The dashed line in the graphs (right) represents the mock treatment. The increase of 9% in dry biomass compared to a formulation without bacterial strain is visualized in FIG. 1A. The increase of 10% in dry biomass compared to a formulation without bacterial strain is visualized in FIG. 1B. The increase of 17% in number of tillers per wheat plant compared to a formulation without bacterial strain is visualized in FIG. 1C.

### Example 3: Increased Grain Yield in Wheat Plants Grown in the Field from Wheat Seeds Treated by a Method According to an Embodiment of the Invention

Per treatment, 1.5 kg winter wheat seeds were coated with a formulation containing the Streptomyces strain A100D6 and a colorant as described in Example 1. Seeds were sown on 4 replicate plots (15 m² plot size) per field location using standard agricultural practices. Sowing density was 300 to 400 seeds per m². Sowing was done from mid to end of October and harvest happened at the end of July. Fertilization was calculated based on soil analysis. 50 kg of phosphorus (P2O5) and 50 kg of potassium (K2O) fertilizers were applied at sowing time. Nitrogen fertilizer was applied at 50 or 80% level at three timings: 80 kg N ha-1 at tillering stage, 60-80 kg N ha-1 at 2-3 node stage and 40 kg N ha-1 at flag leaf stage (100% nutrient level). Harvest was done with a Delta plot combine (Wintersteiger AG, Ried, Austria) and seed yield (grain yield) (kg/ha) was calculated based on the grain yield harvested at each individual plot and considering a seed moisture of 14%. Grain yield was compared with untreated seeds. The results of the wheat plants grown from wheat seeds coated with a formulation containing the Streptomyces strain A100D6 are visualized in FIG. 2.

FIG. 2A visualizes the value in grain yield of winter wheat measured at one location with 95% confidence intervals for coated seeds and untreated seeds. The dashed line in the graphs represent the untreated seeds. Wheat treated with the formulation containing the Streptomyces strain A100D6 and colorant showed an increased yield of 6.5% compared to the untreated seeds.

FIG. 2B visualizes the value in grain yield of winter wheat measured at one location with 95% confidence intervals for coated seeds and untreated seeds. The dashed line in the graphs represent the untreated seeds. Wheat treated with the formulation containing a Streptomyces strain A100D6 and colorant showed an increased yield of 4.9% compared to the untreated seeds.

FIG. 2C visualizes the value in grain yield of winter wheat measured at one location with 95% confidence intervals for coated seeds and untreated seeds. The dashed line in the graphs represent the untreated seeds. Wheat treated with the formulation containing the Streptomyces strain A100D6 and colorant showed an increased yield of 6% compared to the untreated seeds.

FIG. 2D visualizes the value in grain yield of winter wheat measured at one location with 95% confidence intervals for coated seeds and untreated seeds. The dashed line in the graphs represent the untreated seeds. Wheat treated with the formulation containing the Streptomyces strain A100D6 and colorant showed an increased yield of 11.3% compared to the untreated seeds.

### Example 4: Increased Grain Yield in Wheat Plants Grown in the Field from Wheat Seeds Treated by a Method According to an Embodiment of the Invention

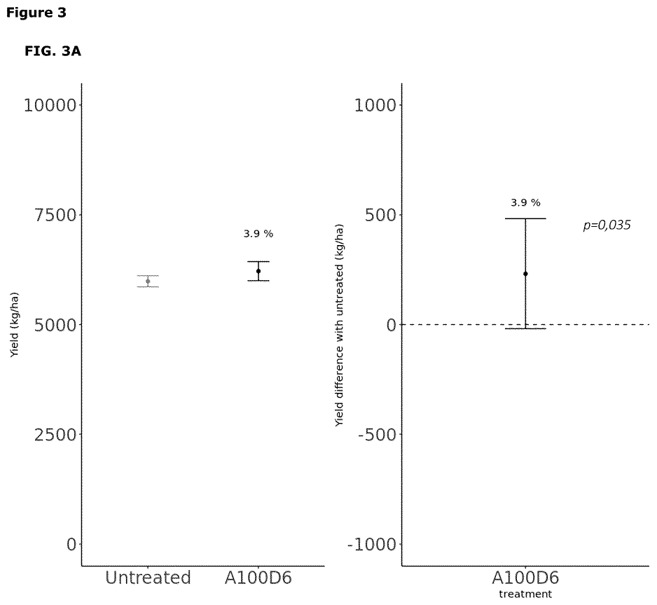

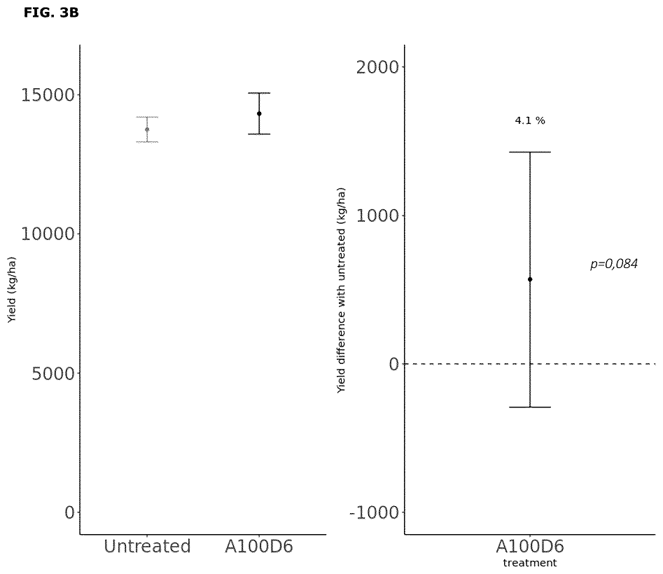

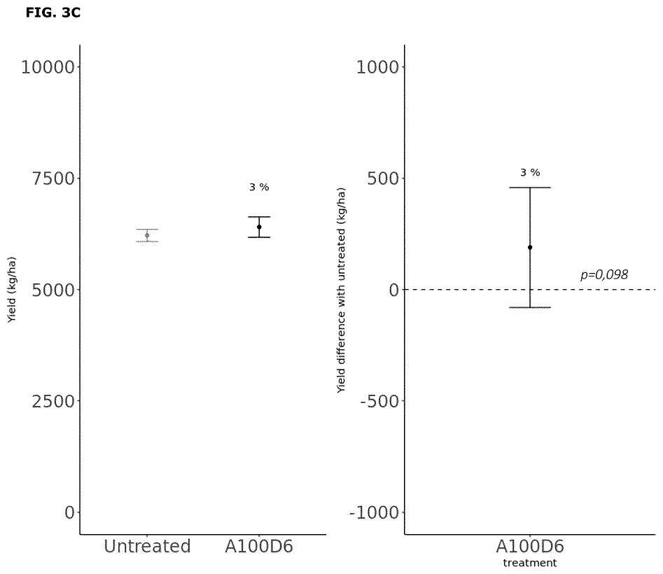

Per treatment, 1.5 kg winter wheat seeds were coated with a formulation containing bacterial spores of the A100D6 bacterial strain and a colorant. Seeds were sown in 4 replicates (11.6 to 18 m² plot size) per field location using standard agricultural practices. Sowing density was 300 to 360 seeds per m². Sowing was done mid October of 2021 and harvest happened the end of July of 2022. Fertilization was calculated based on soil analysis. Nitrogen fertilizer was applied at 70 or 100% level at three timings: 80 kg N ha-1 at tillering stage, 60-80 kg N ha-1 at 2-3 node stage and 40 kg N ha-1 at flag leaf stage (100% nutrient level). Harvest was done with a Delta plot combine (Wintersteiger AG, Ried, Austria) and seed yield (grain yield) (kg/ha) was calculated based on the grain yield harvested at each individual plot and considering a seed moisture of 14%. Grain yield was compared with untreated seeds. The results of the wheat plants grown from wheat seeds coated with a formulation containing spores of the bacterial strain are visualized in FIG. 3.

### Example 5: Beneficial Effects of Bacterial Cells According to an Embodiment of the Invention on Certain Characteristics of Plants

### Iron Availability

The ability to produce different siderophores can help increase iron availability for the plant. Iron is an important micronutrient for several vital processes in plants, such as respiration and photosynthesis. Through chelation, siderophores offer a high-affinity system for the uptake of iron from the environment.

We assessed siderophore production by the A100D6 strain in vitro by using an optimized version of the Blue Agar CAS assay (Louden et al., Journal of Microbiology & amp. 2011, vol. 12; 51-53) originally developed by Schwyn and Neiland. The experiment was carried out on agar plates, in triplicate. The universal siderophore assay is based on chrome azurol S (CAS) and hexadecyltrimethylammonium bromide (HDTMA) used as indicators. The CAS/HDTMA complexes tightly with ferric iron to produce a blue color. When a strong iron chelator such as siderophore removes iron from the dye complex, the color changes from blue to orange. Negative controls (blank samples not containing the A100D6 strain) were included in the assay. Due to the visible halo around its colonies, the A100D6 strain was scored as positive (not shown).

Capability of Coping with Abiotic Stress (Salinity)

Salinity stress is one of the major abiotic stresses threatening sustainable crop production worldwide. The abiotic stress produces various detrimental effects on plants including reduced nutrient mobilization and formation of reactive oxygen species (ROS). Additionally, salinity alters physicochemical properties and reduces the microbial diversity of soil and thus decreases soil health. Salinity-tolerant bacteria can assist the plants in withstanding saline conditions.

Through whole genome analysis, we confirmed the potential of the A100D6 bacterial strain to produce the osmolytes trehalose (otsA, treY, and treZ genes, involved in trehalose biosynthesis). This demonstrates the genetic potential of the A100D6 strain to produce trehalose and thereby enhance its capability to survive extended periods of abiotic stress.

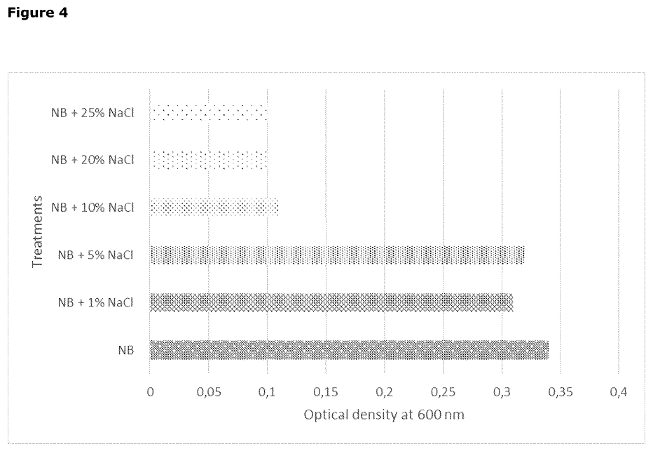

As expected from the genome analysis, this trait was confirmed in vitro. The growth of the A100D6 strain is not affected when in nutrient broth (NB) medium supplemented with NaCl up to 5 wt %. The experiment was carried out in triplicate in 96-well plates. The strain was exposed to the following concentrations of NaCl wt %: 1%, 5%, 10%, 20%, 25%. The microbial growth was assessed by optical density measurements (600 nm) at the following times: 0 h, 3 h, 6 h, 24 h, 48 h, 72 h (FIG. 4). The OD values were compared with control samples including the A100D6 strain growing in optimal conditions (NB with no salt supplements).

### DEPOSIT OF BIOLOGICAL MATERIAL

The purified bacterial strain (the inventors originally proposed the taxonomic designation Streptomyces tendae for the strain, whereas further characterisation indicates that the strain may be more accurately classified as a Streptomyces marokkonensis strain) as taught herein has been deposited at the Polish Collection of Microorganisms (PCM) on 11 Feb. 2022, with the following identification reference given to the deposited material by the depositor: TDC-B-A100D6. Table 1 summarizes the requisite indications relating to this deposited microorganism. For reasons of brevity, this strain is also referred to as strain “TDC-B-A100D6” or strain “A100D6” throughout this specification.

TABLE 1

| | |
|---|---|
| Accession number | B/00365 |
| given by depositary | |
| institution | |
| Identification | TDC-B-A100D6 |
| reference given by | |
| the depositor | |
| Name of depositary | Polish Collection of Microorganisms (PCM) |
| institution | |
| Address of | Institute of Immunology and |
| depositary | Experimental Therapy |
| institution | Polish Academy of Sciences |
| | Ul. Weigla 12 |
| | 53-114 Wroclaw |
| | Poland |
| Date of deposit | 11 Feb. 2022 |
| Depositor | Aphea.Bio NV |
| Address of depositor | Technologiepark 21, B-9052 |
| | Zwijnaarde, Belgium |
| Proposed taxonomic | Streptomyces tendae or |
| designation | Streptomyces marokkonensis |

### SEQUENCE LISTING

Throughout the description and examples, reference is made to the following sequences, as shown in Table 2:

SEQ ID NO: 1: Nucleotide sequence of 16S polynucleotide from strain as deposited under the Budapest Treaty at the Polish Collection of Microorganisms (PCM) under accession no. B/00365 on 11 Feb. 2022 with the following identification reference given to the deposited microorganism by the depositor: TDC-B-A100D6.

TABLE 2

| | SEQ ID | |
|---|---|---|
| Strain | NO | Sequence of 16S polynucleotide (5′-3′) |
| B/00365 | 1 | CACGGAGAGTTTGATCCTGGCTCAGGACGAACGCTGGCGGCGTGCTTAAC |
| | | ACATGCAAGTCGAACGATGAACCACTTCGGTGGGGATTAGTGGCGAACGG |
| | | GTGAGTAACACGTGGGCAATCTGCCCTGCACTCTGGGACAAGCCCTGGAA |
| | | ACGGGGTCTAATACCGGATACTGACCCTCACGGGCATCTGTGAGGTTCGA |
| | | AAGCTCCGGCGGTGCAGGATGAGCCCGCGGCCTATCAGCTTGTTGGTGAG |
| | | GTAATGGCTCACCAAGGCGACGACGGGTAGCCGGCCTGAGAGGGCGACC |
| | | GGCCACACTGGGACTGAGACACGGCCCAGACTCCTACGGGAGGCAGCAGT |
| | | GGGGAATATTGCACAATGGGCGAAAGCCTGATGCAGCGACGCCGCGTGA |
| | | GGGATGACGGCCTTCGGGTTGTAAACCTCTTTCAGCAGGGAAGAAGCGAA |
| | | AGTGACGGTACCTGCAGAAGAAGCGCCGGCTAACTACGTGCCAGCAGCCG |
| | | CGGTAATACGTAGGGCGCGAGCGTTGTCCGGAATTATTGGGCGTAAAGAG |
| | | CTCGTAGGCGGCTTGTCACGTCGGTTGTGAAAGCCCGGGGCTTAACCCCG |
| | | GGTCTGCAGTCGATACGGGCAGGCTAGAGTTCGGTAGGGGAGATCGGAA |
| | | TTCCTGGTGTAGCGGTGAAATGCGCAGATATCAGGAGGAACACCGGTGGC |
| | | GAAGGCGGATCTCTGGGCCGATACTGACGCTGAGGAGCGAAAGCGTGGG |
| | | GAGCGAACAGGATTAGATACCCTGGTAGTCCACGCCGTAAACGGTGGGCA |
| | | CTAGGTGTGGGCGACATTCCACGTCGTCCGTGCCGCAGCTAACGCATTAAG |
| | | TGCCCCGCCTGGGGAGTACGGCCGCAAGGCTAAAACTCAAAGGAATTGAC |
| | | GGGGGCCCGCACAAGCGGCGGAGCATGTGGCTTAATTCGACGCAACGCG |
| | | AAGAACCTTACCAAGGCTTGACATACACCGGAAAGCATTAGAGATAGTGC |
| | | CCCCCTTGTGGTCGGTGTACAGGTGGTGCATGGCTGTCGTCAGCTCGTGTC |
| | | GTGAGATGTTGGGTTAAGTCCCGCAACGAGCGCAACCCTTGTCCCGTGTTG |
| | | CCAGCAAGCCCTTCGGGGTGTTGGGGACTCACGGGAGACCGCCGGGGTCA |
| | | ACTCGGAGGAAGGTGGGGACGACGTCAAGTCATCATGCCCCTTATGTCTT |
| | | GGGCTGCACACGTGCTACAATGGCCGGTACAATGAGCTGCGATACCGCGA |
| | | GGTGGAGCGAATCTCAAAAAGCCGGTCTCAGTTCGGATTGGGGTCTGCAA |
| | | CTCGACCCCATGAAGTCGGAGTCGCTAGTAATCGCAGATCAGCATTGCTGC |
| | | GGTGAATACGTTCCCGGGCCTTGTACACACCGCCCGTCACGTCACGAAAGT |
| | | CGGTAACACCCGAAGCCGGTGGCCCAACCCCTTGTGGGAGGGAGCTGTCG |
| | | AAGGTGGGACTGGCGATTGGGACGAAGTCGTAACAAGGTAGCCGTACCG |
| | | GAAGGTGCGGCTGGATCACCTCCTTT |

1. A method of administering a bacterial strain to a plant, the method comprising:
administering the bacterial strain to the plant, a part thereof, a seed for growing the plant, or a location comprising the plant;
wherein the bacterial strain comprises a 16S polynucleotide having at least 99.34% sequence identity to SEQ ID NO:1.

2. The method according to claim 25, wherein the plant growth feature comprises biomass, number of tillers per plant, yield, or any combination thereof.

3. The method according to claim 2, wherein the biomass, number of tillers per plant, and/or yield of the plant is each independently increased by at least about 2%.

4. The method according to claim 1, wherein the bacterial strain comprises a 16S polynucleotide having at least 99.41% sequence identity to SEQ ID NO: 1.

5. The method according to claim 1, wherein the bacterial strain comprises a 16S polynucleotide as set forth in SEQ ID NO: 1.

6. The method according to claim 1, wherein the bacterial strain is a Streptomyces tendae or Streptomyces marokkonensis strain.

7. The method according to claim 1, wherein the bacterial strain is the strain as deposited under the Budapest Treaty at the Polish Collection of Microorganisms (PCM) under accession no. B/00365 on 11 Feb. 2022.

8. (canceled)

9. The method according to claim 1, the method further comprising administering to the plant, the part thereof, the seed for growing the plant, or the location comprising the plant one or more additional plant-beneficial microorganisms.

10. The method according to claim 27, wherein the agricultural active composition:
comprises one or more agriculturally acceptable auxiliaries, a solvent, a carrier, a surfactant, a sticker, an antifreeze agent, a thickener, a buffering agent, an antifoaming agent, an antioxidant, a preservative, an aroma, a colorant, or a combination thereof;
is a liquid composition, an aqueous composition, a sprayable liquid or a concentrate, a non-liquid composition, or a powder; and/or
comprises the bacterial strain at an amount of at least about 10² CFU/g.

11. The method according to claim 1, wherein
administering the bacterial strain to the plant, the part thereof, the seed for growing the plant, or the location comprising the plant occurs prior to planting the seed, or after planting and before germination and/or
inoculating soil or a plant growth medium with the bacterial strain.

12. (canceled)

13. A composition comprising a plant or part thereof and at least 10 CFU of a bacterial strain comprising a 16S polynucleotide having at least 99.34% sequence identity to SEQ ID NO:1.

14. The composition according to claim 13, wherein the plant part is a seed.

15. The composing according to claim 13, wherein the A bacterial strain is the strain as deposited under the Budapest Treaty at the Polish Collection of Microorganisms (PCM) under accession no. B/00365 on 11 Feb. 2022.

16. (canceled)

17. An agricultural active composition comprising: a bacterial strain comprising a 16S polynucleotide having at least 99.34% sequence identity to SEQ ID NO:1; and wherein the composition:
comprises one or more agriculturally acceptable auxiliaries, a solvent, a carrier, a surfactant, a sticker, an antifreeze agent, a thickener, a buffering agent, an antifoaming agent, an antioxidant, a preservative, an aroma, a colorant, or a combination thereof;
is a liquid composition, an aqueous composition, a sprayable liquid or a concentrate, a non-liquid composition, or a powder; and/or
comprises the bacterial strain at an amount of at least about 10² CFU/g.

18. (canceled)

19. (canceled)

20. The composition of claim 17, wherein the bacterial strain is the strain as deposited under the Budapest Treaty at the Polish Collection of Microorganisms (PCM) under accession no. B/00365 on 11 Feb. 2022.

21. (canceled)

22. (canceled)

23. (canceled)

24. The method according to claim 1 wherein the plant is a monocotyledon, a cereal, wheat, maize, barley, rice, millet, rye, triticale, sorghum, emmer, spelt, einkorn, teff, milo, or oats.

25. The method according to claim 1, wherein the administration improves a plant growth feature of the plant as compared to an otherwise identical plant which has not been administered the bacterial strain thereof.

26. The method according to claim 25, wherein the bacterial strain is the strain as deposited under the Budapest Treaty at the Polish Collection of Microorganisms (PCM) under accession no. B/00365 on 11 Feb. 2022, or a functional mutant thereof, wherein the functional mutant is a bacterial strain directly or indirect obtained by genetic modification of the deposited strain and retaining at least 50% of the activity of the deposited strain on the plant growth feature.

27. The method according to claim 1, wherein administering the bacterial strain thereof to the plant, the part thereof, the seed for growing the plant, or the location comprising the plant comprises administering an agricultural active composition comprising the bacterial strain.

28. The composition of claim 14, wherein the plant seed is coated with the bacterial strain.
